(19) United States
(12) Patent Application Publication
(10) Pub. No.: US 2014/0148615 A1
(43) Pub. Date: May 29, 2014

(54) RECOVERY OF ORGANIC ACID USING A COMPLEX EXTRACTION SOLVENT

(71) Applicants: Dan W. Verser, Menlo Park, CA (US); Gal Mariansky, San Francisco, CA (US); Kevin Carlin, Oakland, CA (US); Erik Gallegos-Westling, San Francisco, CA (US); Jesse James Coutu, San Francisco, CA (US); Timothy J. Eggeman, Lakewood, CO (US)

(72) Inventors: Dan W. Verser, Menlo Park, CA (US); Gal Mariansky, San Francisco, CA (US); Kevin Carlin, Oakland, CA (US); Erik Gallegos-Westling, San Francisco, CA (US); Jesse James Coutu, San Francisco, CA (US); Timothy J. Eggeman, Lakewood, CO (US)

(73) Assignee: ZEACHEM, INC., Menlo Park, CA (US)

(21) Appl. No.: 13/880,286

(22) PCT Filed: Oct. 17, 2011
(86) PCT No.: PCT/US11/56568

§ 371 Date: Jun. 27, 2013

Related U.S. Application Data
(60) Provisional application No. 61/394,295, filed on Oct. 18, 2010.
(60) Provisional application No. 61/510,373, filed on Jul. 21, 2011.

(51) Int. Cl. C07C 51/487; C07C 67/08

(52) U.S. Cl. CPC C07C 51/487; C07C 67/08
USPC 560/265; 562/608

(57) ABSTRACT

A method is disclosed for the recovery of an organic acid from a dilute salt solution in which the cation of the salt forms an insoluble carbonate salt. An amine, C02 and a water immiscible solvent are introduced to the solution to form the insoluble carbonate salt and a complex between the acid and the amine that is soluble in both an aqueous and a solvent phase. The complex is extracted into the solvent phase which is than distilled to recover the acid or an ester of the acid in a concentrated form.

### FIELD OF THE INVENTION

The present invention is related to methods for recovery of organic acids from dilute salt solutions, such as fermentation broths.

### BACKGROUND OF THE INVENTION

Organic acids are valuable products as food and feed ingredients, for example, or as intermediates in the production of other chemicals. For example, organic acids can be chemically converted into alcohols, which can subsequently be converted to olefins. Such a process could be envisioned as the basis for a biorefinery to convert biomass resources into a range of products for the energy and chemical industries.

Many valuable organic acids, such as acetic, lactic and propionic acids, can be produced by fermentation. Holten, Lactic Acid: Properties and Chemistry of Lactic Acid and Derivatives, Verlag Chemie, 1971; Benning a, (1990), A History of Lactic Acid Making: A Chapter in the History of Biotechnology, Kluwer Academic Publishers, London; Partin, L., Heise, W. (1993), in Acetic Acid and Its Derivatives, Agreda, V., Zoeller, J., ed., Marcel Dekker, New York, pp. 3-13; Playne, 1985 Propionic and butyric acids pp. 731-759, In M. Moo-Young (ed.) Comprehensive Biotechnology, vol. 3, Pergamon, Oxford. Using known fermentation methods, such acids can be produced at very high carbon yield from a wide range of biomass resources. However, today almost all organic acids are produced from petrochemicals.

The production of organic acids by fermentation usually requires neutralization of the broth as fermentation proceeds so that it does not become too acidic. Many fermentation reactions operate optimally near neutral pH and failure to maintain proper control of the pH of the fermentation broth can result in inhibition of the fermentation organism. Thus, maintenance of neutral pH is usually carried out by the addition of a base, such as ammonia, NaOH, Ca(OH)₂ or CaCO₃, to the fermenter. However, because the cation of the base combines with the organic acid, the result of such treatment is a dilute salt of the organic acid, such as ammonium acetate, sodium acetate or calcium acetate, and not the free acid itself. Therefore, if it is desired to recover the free acid, it is necessary to convert the organic acid salt back to the free acid. Moreover, these fermentation broths are quite dilute. Thus, an efficient recovery method with respect to both the acidification issue and the dilution issue is desirable.

Many methods have been proposed to address this problem. Among the simplest methods is the addition of a strong mineral acid, such as sulfuric acid, to the broth containing the organic acid salt. Because such acids are much stronger than organic acids, their addition shifts the ionic equilibrium so that essentially all of the organic acid salt is converted to the free acid. However, the strong acid is itself simultaneously converted to a salt. If the salt is not useful it can be disposed of, but this is often an economic and environmental burden since the byproduct salt is produced in an equal molar amount as the organic acid.

Other methods have been proposed to recover the organic acid from the dilute salt solution. One of the more interesting is the use of an amine to convert the alkaline metal salt to an organic salt. For example, Urbas, U.S. Pat. No. 4,405,717, incorporated herein by reference in its entirety, describes the use of tributyl amine (TBA) and CO₂ to convert a dilute calcium salt to an insoluble CaCO₃ and a water-soluble organic complex of TBA and acetic acid at very high yield. Urbas suggests the extraction of the TBA acid complex from the dilute aqueous solution and then the concentration and “cracking” or thermal decomposition of the recovered organic complex to regenerate the TBA and the acetic acid. However this method requires separating the solvent from the amine which is energy intensive. For the extraction step Urbas teaches away from the use of solvents like alcohols that reacts with the acid and recommends the use of chloroform which is problematic because of its toxicity to the environment.

Verser et al (U.S. Patent Publication No. 2005/0256337), incorporated herein by reference in its entirety, describe the recovery of the acid from the extracted TBA acid complex by forming its ester directly from the extract. However, the esterification reaction is conducted in the presence of the amine and is fairly slow.

Similarly, Verser et al. (U.S. Pat. No. 6,509,180, incorporated herein by reference in its entirety, describes the production of ethanol from acetic acid produced by fermentation. The acetic acid is reacted with an amine to form an acid/amine complex, which is then thermally cracked to release the acid. The released acid is then esterified to form alcohol. Similarly, Verser et al. (U.S. Patent Publication NO. 20080193989), incorporated herein by reference in its entirety, also teaches forming a complex between an organic acid and an amine.

Mariansky et al (U.S. Patent Publication No. 2009/0281354 A1), incorporated herein by reference in its entirety, describe the recovery of the acid from a TBA acid complex by thermally cracking the complex at high temperature while extracting the TBA into a solvent phase. The acid, now in the protonated form, can be recovered from the dilute aqueous solution by a second extraction. This recovery process requires two extraction trains, one of them operated at high temperature and high pressure, which make this process too costly for a commercial process.

A number of other processes have been proposed for recovery of organic acids from dilute acid salt solutions. Thomas et al published U.S. Patent Application 2006/0024801 A1 where they reacted the salt with a low molecular amine, concentrate by evaporation, replace the low molecular amine with a high molecular amine in an extractive distillation column and distill the acid from the high molecular amine. Similar to Mariansky et al., this process requires two extraction trains and is too costly for a commercial process.

King, et al. U.S. Pat. No. 5,068,180, describes a method to recover the acid by adsorbing it on a strongly basic ion exchanger and desorbing using a light amine or ammonia solution. The resulting salt is than thermally cracked to by evaporating the amine and water away from this acid. This method offers only limited recovery from salt solutions and only works for non-volatile acids (e.g. lactic acid)

Baniel et al. U.S. Pat. No. 6,087,532, describe an extraction method to recover the acid from a salt solution by combining it with high molecular weight amine and CO₂. The carbonic acid resulting from the CO₂ dissolving in the water acidifies a portion of the acid salt which is than extracted into the amine phase. Because most organic acids are stronger acids than carbonic acid, only a small portion of the acid salt is acidified and therefore the extraction coefficient is very low which necessitate a high solvent to feed ratio for high recovery rates of the acid.

Datta et al. U.S. Pat. No. 5,723,639 teaches a method where a light amine or ammonia and a light alcohol are combined with the salt solution; the mixture is heated in the presence of a catalyst and subjected to pervaporation with hydrophilic membrane. However the reaction rates and conversion are too low to be practical in a commercial process.

Thus, while the prior art discloses methods for recovering organic acids from fermentation broths, such methods require two separate extraction loops or high solvent to feed or high energy use, or all of these combined. Thus, a need exist for a simple method that provides a process with low capital cost and low energy use to recover the acid in a concentrated form from the dilute acid salt. The present invention satisfies this need and provides other advantages as well.

### BRIEF DESCRIPTION OF THE DRAWINGS

FIG. 1 is a flow diagram illustrating the reaction and purification steps, as well as the flow streams in the CCE/LLE system of the claimed process.

FIGS. 2A and 2B are alternative flow diagrams illustrating the recovery system in the claimed process.

FIG. 3 is a flow diagram illustrating a CCE/LLE system containing two CCE/separation steps.

FIGS. 4A and 4B illustrate a multi-step CCE/LLE system (4A) and a recovery system for recovering the acid in the ester form. FIG. 4C illustrates an alternative recovery system when an alcohol, such as 1-hexanol or 1-pentanol, is used as the solvent, or as part of the solvent.

FIG. 5 illustrates the kinetics of % conversion of the acid salt to an acid/amine complex in a Combined Carbonation and Extraction (CCE) batch reactor.

FIG. 6 illustrates the effect of temperature on the conversion of the organic acid salt into an acid/amine complex.

FIG. 7 is a schematic diagram of a pilot unit assembled to generate design data for a commercial, counter-current, multi-stage CCE and LLE process.

FIG. 8 illustrates the percent recovery by steam stripping of tributylamine from raffinate-solids slurry exiting the CCE reaction.

FIG. 9 illustrates the separation of the organic acid by distillation from the extract stream leaving the CCE reaction.

FIG. 10 illustrates the separation of the organic acid by distillation from the extract stream leaving the CCE reaction.

FIG. 11 illustrates the recovery of acid/amine complex from CCE-generated raffinate using co-current extractions.

FIG. 12 is a McCabe-Thiele diagram illustrating the percent recovery of the organic acid from the aqueous phase into the solvent phase resulting from a countercurrent five equilibrium-stage LLE cascade.

FIG. 13 illustrates the extraction coefficient of organic acid vs. the weight % of acid in the raffinate resulting from five co-current LLE's using a salt feed produced by fermentation of sugars.

FIG. 14 illustrates the effect of temperature on the efficiency of acid extraction.

FIG. 15 illustrates the saponification rates of pentyl acetate to pentanol and an acetic acid salt using various bases.

### SUMMARY OF THE INVENTION

The present innovation provides a method for recovery of organic acids from their salt solutions by reacting the salts with an amine and CO₂ (carbonation) to form an insoluble carbonate salt and an acid/amine complex, which is soluble in the feed salt solution, while simultaneously extracting the resulting acid/amine complex into a solvent phase. The acid can then be recovered from the solvent phase by distilling it away from the amine and solvent which can then be recycled back to the combined carbonation and extraction step (CCE).

The advantage of combining the carbonation reaction with the extraction is to further drive the carbonation reaction to completion and allow a higher concentration feed stream to be processed by removing the reaction product amine/acid complex from the aqueous reaction phase.

One embodiment of the present invention is a method for recovering an organic acid from a salt solution that comprises an organic acid salt, the cation of which forms an insoluble carbonate salt. The method includes introducing an amine, carbon dioxide and a solvent to the salt solution to form an insoluble carbonate salt phase, an aqueous phase, and a solvent phase containing an acid/amine complex. The method further comprises recovering the acid from the solvent phase.

The acid being recovered can be any acid having a boiling point lower than the boiling point of the solvent. In one embodiment, the acid is a carboxylic acid. In one embodiment, the acid is acetic acid, lactic acid, propionic acid, butyric acid, succinic acid, citric acid, 3-hydroxypropionic acid, glycolic acid or formic acid.

In one embodiment, the aqueous salt solution is a fermentation broth. The broth can be concentrated and/or filtered. The concentration of acid in the fermentation broth can be at least about 10%, or at least about 20%.

A further embodiment of the present invention is a method for recovery of an organic acid from a salt solution in which the cation of the organic acid salt is Ca, Zn, Ba or Mg. The amine introduced into the salt solution to form the acid/amine complex has a solubility of less than about 0.5% at room temperature. The solubility of the resultant acid/amine complex in the salt solution is more than about 0.5% on an acid basis. In one embodiment, the amine is tributylamine, dicyclohexyl methyl amine, di-isopropyl ethyl amine, tripropylamine, or mixtures thereof.

In a further embodiment, the solvent has a boiling point that is higher than the boiling point of the acid. The solubility of the solvent in the salt solution is at least about 0.05%. In one embodiment, the solvent is polar. In another embodiment, the solvent has a distribution coefficient (Kd) with respect to the acid of at least about 0.5. In one embodiment, the solvent is selected from the group consisting of alcohol, a ketone, an ester, a hydrocarbon, an organophosphate, an amide, a higher molecular weight organic acid, and mixtures thereof. In a further embodiment, the solvent is selected from the group consisting of n-octanol, n-hexanol, n-pentanol, n-butanol, 2-ethyl hexanol, 2-ethyl-2-hexanol, 2-ethyl-hexyl acetate, tri-octyl-phosphine oxide, tri-butyl phosphate, heptane, 2-octanone, hexanoic acid, N,N-di-n-butylformamide, decanoic acid, decane, octyl acetate, and mixtures thereof.

In a further embodiment, the solvent comprises an enhancer that is extractable from the aqueous phase, and has at least one property selected from the group consisting of a) being highly polar; and b) having the ability to hydrogen bond with the acid. In another embodiment, the enhancer forms an azeotrope with water having a boiling point lower than the boiling point of the solvent and the acid. In one embodiment, the enhancer is selected from the group consisting of an alcohol, an organochloride, an organophosphate, an amide, and mixtures thereof.

The acid is recovered from the solvent phase. One embodiment of the present invention comprises separating at least a portion of the solvent phase from at least a portion of the aqueous phase, and recovering the acid from the solvent phase. The insoluble carbonate salt is also separated from the aqueous phase. In a further embodiment, the separated solvent phase is distilled to produce an acid-containing distillate and an acid-depleted bottoms fraction. In another embodiment, water is removed from the solvent phase by distilling the water as a heterozygous azeotrope with the enhancer.

The present invention offers advantages of efficiency with regard to cost since the amine and the solvent need never be separated and are recycled back into the process. In one embodiment, the bottoms fraction from the distillation of the separated solvent phase is combines with the separated aqueous phase, resulting in formation of a new solvent phase and a new aqueous phase. As a result of this new phase formation, acid/amine complex in the separated aqueous phase is extracted into the new solvent phase. This new solvent phase is then added into the aqueous salt solution.

In one embodiment of the present invention, the method comprises removing residual solvent and amine from the aqueous phase and combining them with the bottoms fraction. In one embodiment, removal of the solvent and amine from the aqueous phase comprises the use of steam stripping.

One embodiment of the present invention is a method for the recovery of an organic acid from an aqueous salt solution, wherein the cation of the salt forms an insoluble carbonate salt. The method includes introducing an amine, carbon dioxide and a solvent to the aqueous salt solution to form a mixture having an insoluble carbonate salt phase, an aqueous phase, a solvent phase and an acid/amine complex. The method further includes recovering the acid from the solvent phase to form an acid-depleted solvent phase. The aqueous phase and the acid-depleted solvent phase are combined, and as a result, acid/amine complex in the aqueous phase is transferred to the acid-depleted solvent phase. In this manner, an acid-depleted aqueous phase and an acid-enriched solvent phase are formed. The method further includes separating the acid-depleted aqueous phase and the acid-enriched solvent phase and introducing the acid-enriched solvent phase to the aqueous salt solution.

A further embodiment of the present method is to recover acid in the form of an ester. In such an embodiment, the acid is reacted with an alcohol to form an ester. This embodiment includes a method to recover an ester from a solution of an organic acid salt, wherein the cation of the salt forms an insoluble carbonate salt. The method includes introducing an amine, carbon dioxide, a solvent and an alcohol to the organic acid salt solution to form a mixture having an insoluble carbonate salt phase, an aqueous phase, a solvent phase and an acid/amine complex. The acid is recovered from the solvent phase, and reacted with an alcohol to form an ester. In this embodiment, the alcohol can be the solvent or an enhancer and can be recovered in the same stream as the acid. Alternatively, the alcohol can be added after the acid is recovered from the solvent phase.

The alcohol introduced into the aqueous salt solution can have a boiling point lower than that of the solvent and the amine. In one embodiment, the alcohol introduce into the aqueous salt solution is selected from the group consisting 1-butanol, 2-butanol, 1-pentanol, and 1-hexanol.

In a further embodiment, the ester is hydrogenated to form the alcohol introduced to the aqueous salt solution and an alcohol product. In one embodiment, the alcohol product is ethanol. In another embodiment, the alcohol product is propanol. In a further embodiment, the alcohol product is a mixture of ethanol and propanol.

### DETAILED DESCRIPTION OF THE INVENTION

The present invention provides methods for the recovery and separation of organic products (e.g., carboxylic acids, esters, etc.) from a solution of organic acid, such as a fermentation broth. The organic acids are typically in the form of salts formed from the reaction of the organic acid and a base, the latter being added to the fermentor to neutralize the acid during fermentation. For example, if a fermentation producing acetic acid is neutralized with calcium carbonate, the resulting organic acid salt produced in fermentation will be calcium acetate.

The organic acid salt can then be reacted with an amine, such as a tertiary amine, and carbon dioxide, and solvent to form an acid/amine complex and an insoluble carbonate salt. For example:

Ca(Ac)2+H2O+CO2+2TBA=>2TBA:HAc+CaCO3

The resulting acid/amine complex will be distributed between the aqueous phase and the solvent phase. The solvent phase can then be separated from the mixture, and the acid recovered from the solvent phase using techniques known in the art.

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The methods disclosed herein comprise a counter-current process, in which the incoming organic acid salt solution is treated in a series of steps such that, the organic acid can be recovered as a free acid, or as an ester of the acid. The amine and the solvent need not be separated from one another, thereby reducing the energy cost of the process. Furthermore, the majority of the solvents, and other components used in the process, are recycled back into the process for re-use, thereby reducing the cost of the process as well as its environmental impact.

The process described herein can comprise two systems. The first is a combined carbonation and extraction (CCE) and a liquid-liquid extraction (LLE) system (CCE/LLE system). In this system, acid in the incoming organic acid salt solution is reacted with an amine and carbon dioxide to form an acid/amine complex and an insoluble carbonate salt. The insoluble carbonate salt is separated and optionally recycled back to the fermenter as a base, and the acid/amine complex is extracted into the solvent leaving behind an acid depleted aqueous phase. The second system is a recovery system in which the acid is recovered from the solvent loaded with the acid/amine complex, and the acid-depleted amine and solvent are returned to the CCE/LLE system, where they are utilized in the recovery of more incoming acid.

Each system of the process comprises a series of steps, which themselves comprise one or more functional operations that can be conducted using certain devices. For example, introduction of the organic acid salt solution, CO₂, amine and solvent occurs in a CCE reactor. Such a reactor is, for example, a mixer. The resulting mixture is then passed from the reactor to a separation device, such as, for example, a decanter, for separation of the various phases formed in the CCE reactor. According to the methods disclosed herein, reaction of the organic acid, CO₂, amine and solvent in the CCE reactor, and separation of the resultant phases, are referred to as one step.

One embodiment of the present invention is a method for recovering a product from a dilute solution of an organic acid salt. The aqueous salt solution comprises a salt of the organic acid, the cation of which forms an insoluble carbonate salt. The method comprises introducing an amine, CO₂, and a solvent to the aqueous salt solution to form a mixture comprising an insoluble carbonate salt phase, an aqueous phase, a solvent phase and an acid/amine complex. The method further comprises recovering the product from the solvent phase.

In one embodiment of the present invention, the product recovered is an organic acid. Methods of the present invention can be used to recover any organic acid having a boiling point lower than that of the solvent. In one embodiment, the organic acid recovered using methods of the present invention is selected from the group consisting of: acetic acid, lactic acid, propionic acid, butyric acid, succinic acid, citric acid, 3-hydroxypropionic acid, glycolic acid, or formic acid.

Methods of the present invention are particularly suited to the recovery of products produced by fermentation. In various embodiments, the fermentation medium includes carbohydrate substances, non-carbohydrate substances, and mixtures thereof. Carbohydrate in the fermentation medium can be obtained from biomass, which can include, but is not limited to, herbaceous matter, agricultural residue, forestry residue, municipal solid waste, waste paper, pulp and paper mill residue. Biomass can also be selected from the group consisting of trees, shrubs, grasses, wheat, wheat straw, wheat midlings, sugar cane bagasse, corn, corn husks, corn kernel, corn fiber, municipal solid waste, waste paper, yard waste, branches, bushes, energy crops, fruits, fruit peels, flowers, grains, herbaceous crops, leaves, bark, needles, logs, roots, saplings, short rotation woody crops, switch grasses, vegetables, vines, sugar beet pulp, oat hulls, hard woods, wood chips, intermediate streams from pulping operations and soft woods, and in a preferred embodiment, is selected from the group consisting of trees, grasses, whole plants, and structural components of plants.

The biomass can be pre-treated prior to fermentation. For example, if an agricultural product such as corn is used as a carbohydrate source, the corn can be ground to produce corn meal and/or oil for recovery. In one embodiment, the biomass is hydrolyzed to produce carbohydrate prior to fermentation. In one embodiment, the hydrolysis is enzymatic hydrolysis. In one embodiment, the hydrolysis is chemical hydrolysis.

Fermentation can be conducted using a homofermentative microorganism or a heterofermentative microorganism, depending on the desired final products. In one embodiment, the fermentation is conducted using a microorganism selected from the group consisting of homoacetogenic microorganisms, homolactic microorganisms, propionic acid bacteria, butyric acid bacteria, succinic acid bacteria and 3-hydroxypropionic acid bacteria. In one embodiment, the fermentation is conducted using a microorganism that produces acetate as the primary end product of metabolism. In another embodiment, the fermentation is conducted using a microorganism that produces propionate and acetate as the primary end products of metabolism. In one embodiment, the microorganism is of a genus selected from Clostridium, Lactobacillus, Moorella, Thermoanaerobacter, Propionibacterium, Propionispera, Anaerobiospirillum, and Bacteriodes. In other embodiments, the microorganism is of a species selected from Clostridium formicoaceticum, Clostridium thermoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola.

In one embodiment of the present invention, the fermentation includes converting the carbohydrate source into acetic acid, acetate, lactic acid, lactate, propionic acid, propionate, or mixtures thereof by fermentation. In a further embodiment, the lactic acid, lactate, or mixtures thereof, are at least partially converted into acetic acid, acetate or mixtures thereof by fermentation. The lactic acid fermentation can be homolactic fermentation accomplished using a microorganism of the genus Lactobacillus. Alternatively, the carbohydrate source can be converted into lactic acid, lactate, acetic acid, acetate or mixtures thereof in an initial fermentation using a bifido bacterium.

As noted, reaction of the cation of the organic acid salt with carbonic acid from the CO₂ results in formation of an insoluble carbonate salt. Without being bound by theory, it is believed that precipitation of the cation in the form of the insoluble carbonate salt drives formation of the acid/amine complex, thereby increasing the efficiency of recovery of the acid. Thus, any cation that is capable of forming an insoluble carbonate salt can be used in the present method. Suitable cations include, for example, Ca, Zn, Ba and Mg. In a preferred embodiment, the cation is Ca.

The present method utilizes an amine in order to form an acid/amine complex. Any amine that is capable of forming a complex with the organic acid being recovered can be used in methods of the present invention provided the acid/amine complex is at least slightly soluble in water and can be extracted into the solvent. Without being bound by any particular theory, amines useful for practicing the present invention are those that form an acid/amine complex that is soluble in the aqueous feed solution. In one embodiment, the solubility of the acid/amine complex in the dilute aqueous solution is greater than about 0.5% on the acid basis. The amine itself does not need to be soluble in water and is preferably only slightly soluble in water. In one embodiment, the solubility of the amine in water is less than about 0.5%, or less than about 0.1%, at room temperature.

In one embodiment, the amine is a tertiary amine. In one embodiment, the amine is selected from the group consisting of tributylamine, dicyclohexyl methyl amine, di-isopropyl ethyl amine, tripropylamine, and mixtures thereof.

Depending on the acid being recovered and the amine chosen for the process, the acid/amine complex will have varying coefficients of solubility and extractability. Thus, the choice of solvent will be affected by the amine used in, and the acid recovered by, the present method. Any solvent can be used so long as it works with the chosen amine to effect recovery of the acid from the solvent phase. Preferred solvents are those having properties that improve the yield and efficiencies of the disclosed method. For example, in one embodiment, the solvent has a boiling point that is higher than the boiling point of the acid. In a further embodiment, the solvent is polar. In yet a further embodiment, the solubility of the solvent in the aqueous salt solution is at least about 0.05%.

As has been noted, the disclosed method is based on the formation of an acid/amine complex and the interaction of the acid/amine complex with the solvent. In particular, the method is based on the solubility of the complex in the aqueous salt solution in relation to the solubility of the complex in the solvent. One useful way of evaluating the relative solubilities of the acid/amine complex is by the distribution coefficient (Kd) of the acid between the solvent and the aqueous solution. As used herein, the distribution coefficient is the ratio of the percent mass of acid in the solvent compared to the percent mass of acid in the aqueous solution.

Mathematically, the distribution coefficient is defined as follows:

$$Kd = \frac{\% mass of acid in solvent}{\% mass of acid in aqueous}$$

Use of this coefficient allows the choice of any acid, amine and solvent combination, so long as the combination has a favorable distribution coefficient. In one embodiment, the solvent has a distribution coefficient of at least about 0.5. In one embodiment, the solvent has a distribution coefficient of at least about 0.75. In one embodiment, the solvent has a distribution coefficient of at least about 1.0.

The properties of certain types of solvents make them particularly useful for practicing the disclosed methods. Moreover, the inventors have found that while the solvent can be a single chemical, a mixture of various chemicals having the properties disclosed herein can also be used. Thus, in one embodiment the solvent is selected from the group consisting of an alcohol, a ketone, an ester, a hydrocarbon, an organophosphate, an amide, a higher molecular weight organic acid, and mixtures thereof. In general high molecular weight organic acids contain at least 8 carbon atoms, at least 9 carbon atoms or at least 10 carbon atoms. In one embodiment, the solvent is selected from the group consisting of n-octanol, n-hexanol, n-pentanol, n-butanol, 2-ethyl hexanol, 2-ethyl-2-hexanol, 2-ethyl-hexyl acetate, tri-octyl-phosphine oxide, tri-butyl phosphate, heptane, 2-octanone, hexanoic acid, N,N-di-n-butylformamide, decanoic acid, decane, octyl acetate, and mixtures thereof.

The inventors have also found that the yield of acid may be improved by the adding an enhancer to the solvent. Useful enhancers are those that can be extracted from the aqueous phase. In one embodiment, the enhancer is extractable from the aqueous phase and has at least one property selected from the group consisting of a) being highly polar; and b) having the ability to hydrogen bond with the acid. In one embodiment, the enhancer forms an azeotrope with water having a boiling point lower than the boiling point of the solvent and the acid. In one embodiment, the enhancer is selected from the group consisting of an alcohol, an organochloride, an organophosphate, an amide, and mixtures thereof.

As noted above, the cation reacts with carbonic acid formed by the CO₂, resulting in the formation of an insoluble salt. Precipitation of this salt is believed to improve the efficiency of the process since removal of the cation from the reaction mixture “drives” formation of the acid/amine complex. Thus, in one embodiment, the insoluble salt is separated from the reaction mixture. In a particular embodiment, the insoluble salt is separated from the aqueous phase. Any method that separates the insoluble salt from the reaction mixture or the aqueous phase can be utilized. Methods of separation are known to those skilled in the art and include, but are not limited to, for example, gravity separation, filtration, centrifugation, and combinations thereof.

Based on the afore-mentioned description of the disclosed process, it will be appreciated that following introduction of the amine, carbon dioxide and a solvent to the aqueous phase, both the aqueous phase and the solvent phase can comprise acid/amine complex. According to the present invention, methods for recovering the acid from the solvent phase and the aqueous phase are disclosed herein. For example, in one embodiment, at least a portion of the solvent phase is separated from at least a portion of the aqueous phase to form a separated solvent phase and a separated aqueous phase. As used herein, at least a portion of the solvent phase means at least 70%, at least 80%, at least 90%, or at least 95% of the solvent present in the mixture. Similarly, as used herein, at least a portion of the aqueous phase means at least at least 70%, at least 80%, at least 90%, or at least 95% of the aqueous phase present in the mixture. Separation of theses phases can be achieved using techniques known to those skilled in the art and include, but are not limited to, for example, gravity separation, centrifugation and combinations thereof. For example, separation of the solvent phase from the aqueous phase can be accomplished using a decanter.

In a further embodiment, the acid is distilled from the separated solvent phase to produce an acid-containing distillate and a bottoms fraction. As used herein, the bottoms fraction is what remains in a distillation vessel once at least some of the desired product has been removed in the vapor fraction of the distillation. Suitable methods of distillation are known to those skilled in the art.

According to methods of the present invention, the acid can recovered as a free acid, or it may be recovered in the form of an ester. In one embodiment, the acid-containing distillate comprises free acid. In a further embodiment, the distillate is passed to another column where the free acid in the distillate is reacted with alcohol in the distillate to form an ester.

In a further embodiment, the acid-containing distillate is passed to another column, and light components present in the acid-containing distillate are distilled overhead. In such an embodiment, the organic acid is removed as the bottoms fraction resulting in very pure acid. In one embodiment, acid produced by the disclosed methods has a purity of at least 95%, at least 96%, at least 97%, at least 98%, at least 99% or at least 99.9%.

In a further embodiment, the overhead distillate containing the light components is combined with the separated solvent phase.

In methods of the present invention in which the acid is recovered in the form of an ester instead of a free acid, the acid is reacted with an alcohol to form the ester. In one embodiment, the acid in the separated solvent phase is reacted with the solvent to form an ester. In another embodiment, the acid is reacted with the enhancer to form an ester. It should be noted that in such embodiments, reaction of the solvent, or the enhancer, with the acid will result in depletion of solvent, or enhancer, from the systems. Thus, in such embodiments, it is necessary to replace the solvent, or enhancer, being lost to ester formation. As an alternative to replacing the lost solvent, or enhancer, with new solvent, or enhancer, the ester can be hydrogenated to form the original alcohol used as the solvent, or the original enhancer, and a new alcohol product. For example if the acid is acetic acid and the alcohol used as the solvent is hexanol, the ester produced will be hexyl acetate, the hydrogentation of which will produce hexanol and ethanol.

In another embodiment, an alcohol other than the solvent or the enhancer is added into the process and the acid is reacted with this alcohol.

While, for illustrative purposes, the disclosed methods have been described with regard to recovering an organic acid, it should be understood that the methods disclosed herein can be used to simultaneously recover more than one organic acid. For example, if propionic acid bacteria are used to ferment biomass, the resulting fermentation products will include propionic acid, propionate salt, acetic acid, acetate salt, and mixtures thereof. The disclosed methods can be applied to such a mixture to recover purified propionic acid, acetic acid and mixtures thereof. Thus, one embodiment of the present invention is a method to obtain more than one organic acid from a solution of organic acids, such as a fermentation broth. In one embodiment, the organic acids are obtained as a mixture.

Moreover, as has been described, the organic acids can be recovered as esters. Thus, one embodiment of the present invention is a method to recover more than one ester of an organic acid from a solution of organic acids, such as a fermentation broth. In one embodiment, the esters are obtained as a mixture. Furthermore, it should be appreciated that such esters can be further treated to produce alcohol products. For example, application of the disclosed methods to a fermentation broth produced using propionic acid bacteria will result in production of propionic acid and acetic acid. Esterification of these acids to a solvent, such as hexanol, would yield the propionate and acetate esters, hexyl proprionate and hexyl acetate. Hydrogenation of such esters would regenerate the original hexanol solvent, and yield propanol and ethanol. Depending on the methods used, such alcohols can be recovered as individual alcohols, or as a mixture.

One advantage of the present invention is that various process streams are re-introduced into the process, thereby increasing the overall efficiency of the recovery process. For example, solvent containing amine in the bottoms fraction obtained from distillation of the separated solvent phase can be reused by combining it with the separated aqueous phase resulting in the formation of a second solvent phase and a second aqueous phase. As a further consequence of this phase separation, at least some of any acid/amine complex in the second aqueous phase is extracted into the second solvent phase.

In a further embodiment, at least a portion of the second solvent phase produced by mixing the bottoms fraction from the distillation with the separated aqueous phase, is removed from the second aqueous phase and mixed with the aqueous salt solution.

As has been previously discussed, the amine, acid/amine complex and solvent each can have at least some solubility in water. Consequently, the separated aqueous phase can contain residual amounts of acid, amine, acid/amine complex and solvent. Thus, in one embodiment of the present invention, the residual solvent and amine are removed from the separated aqueous phase. Any suitable technique can be used to remove the amine, solvent and mixtures thereof, from the separated aqueous phase. In one embodiment, amine, acid/amine complex and/or solvent are removed from the separated aqueous phase using at least one technique selected from the group consisting of gravity separation, centrifugation, distillation, and steam stripping. In a further embodiment, the amine and/or solvent removed from the separated aqueous phase are combined with bottoms fraction obtained from distillation of the separated solvent phase.

As previously described, the acid is recovered from the separated solvent phase. In order to increase the purity of the recovered acid, other components present in the separated solvent phase can be removed prior to recovery of the acid. For example, as has been discussed, the solvent stream can also comprise residual water or an enhancer. In one embodiment, residual water is removed from the separated solvent phase by distilling the water as a heterogeneous azeotrope with the enhancer. Such distillation can be performed prior to, or concomitant with recovery of the product.

A further embodiment of the present invention is a method for recovering an organic acid from an aqueous salt solution. The aqueous salt solution comprises a salt of the organic acid, the cation of which forms an insoluble carbonate salt. The method comprises introducing an amine, carbon dioxide and a solvent to the dilute salt solution to form a mixture comprising an insoluble carbonate salt phase, an aqueous phase, a solvent phase and an acid amine complex. The method further comprises recovering the acid from the solvent phase to form an acid-depleted solvent phase. The method further comprises combining the acid-depleted solvent phase with the aqueous phase so that the acid/amine complex in the aqueous phase is transferred to the acid-depleted solvent phase to form an acid-depleted aqueous phase and an acid-enriched solvent phase. The method further comprises introducing the acid-enriched solvent to the aqueous salt solution.

The methods disclosed herein are general methods for the efficient recovery of a product from an aqueous salt solution. Such methods are applicable to both batch processes and continuous processes. Thus, in one embodiment, recovery of a product from an aqueous salt solution using the methods disclosed herein is performed as a batch process. As used herein, a batch process is a process that utilizes a finite amount of starting material. For example, according to the present invention, a batch process would be performed starting with a discrete amount of aqueous salt solution, the acid would be recovered, and no further aqueous salt solution would be added during the process.

In one embodiment, recovery of a product from an aqueous salt solution using the methods disclosed herein is performed as a continuous process. As used herein, a continuous process is one in which a feed material is introduced into the process systems in a periodic or continuous manner. Thus, for example, according to the present invention, in a continuous process, aqueous salt solution would be continuously, or periodically, added into the process.

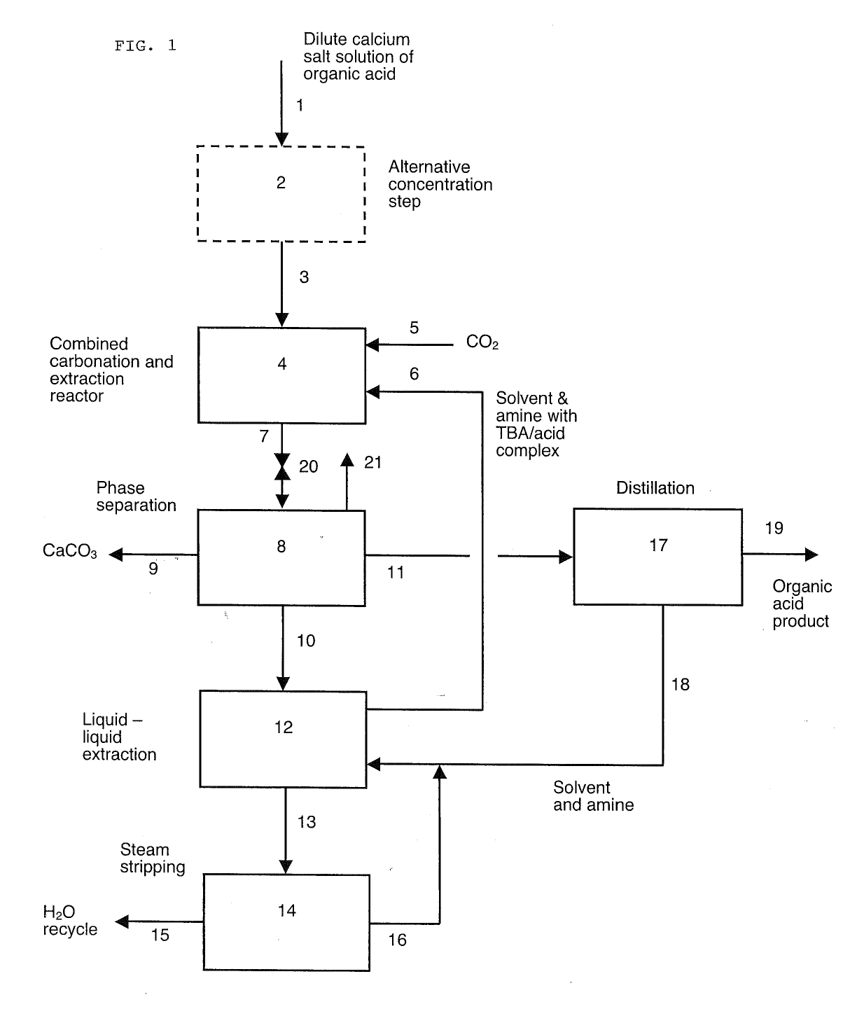

To help further clarify the invention, the present invention is exemplified with reference to FIGS. 1-4.

Combined Carbonation & Extraction (CCE) with Liquid-Liquid Extraction (LLE) System

The CCE/LLE system of the disclosed process is exemplified with reference to FIG. 1. Stream 1 is a dilute solution comprising a calcium salt of an organic acid. This dilute organic salt solution can come from any source such as a recycle stream from other processes such as the production of cellulose acetate or from a fermentation broth. In Step 2, the dilute salt solution can optionally be concentrated. Any process that concentrates Stream 1 can be used for Step 2 including, for example, evaporation or reverse osmosis. Concentration of the feed stream provides the advantage that the water flow in the process systems can be reduced, thus making downstream equipment smaller and less costly. If the feed stream is concentrated, the concentrated solution (Stream 3) is then fed into a carbonation/extraction reactor. If no concentration step is used, Stream 1 fed directly into the carbonation/extraction reactor.

Device 4 is a pressurized carbonation/extraction reactor. The reactor can be any type useful for the intended purpose. For example, the reactor can be a continuous stirred-tank reactor (CSTR), a batch reactor or a plug flow reactor. In the reactor, the incoming stream (Stream 1 or 3) is introduced to a solvent, an amine and carbon dioxide (CO₂) under pressure. Preferably the solvent and amine mixture is provided from a subsequent step in the process (Stream 6), and may optionally contain some of the acid/amine complex, as long as the amine is not saturated with the acid. CO₂ is supplied to the reactor through Line 5. Preferably the reactor is operated at a pressure above atmospheric pressure to enhance the solubility of the CO₂ in the mixture. The pressure in the reactor can be between 25 psig and 500 psig, and preferably about 250 psig. The amount of CO₂ in the reactor is at least in molar proportion required for the carbonation reaction. Preferably the amount of CO₂ is at least twice the amount required for the carbonation reaction. Excess CO₂ can be released at a later point in the process and captured and recycled.

The temperature of the reactor can be adjusted to any suitable temperature. For example, the reactor can be maintained at ambient temperature. However, it may also be useful to use a colder temperature, since this increases the solubility of the CO₂, which has a positive effect on the reaction.

Once all of the components have been introduced into the reactor, the contents of the reactor are mixed. In the reactor, the cation of the organic salt reacts with carbonic acid formed by the CO₂, yielding calcium carbonate and the free acid. The free organic acid reacts with the amine to form an acid/amine complex. Thus, following mixing of all of the components, a mixture is obtained that comprises the following three phases: an insoluble carbonate salt solid, an aqueous phase containing un reacted acid salt and acid/amine complex, and a solvent phase containing acid/amine complex. It should be noted that the CO₂ introduced into the reactor is preferably dissolved in the aqueous and solvent phases. This will depend on the pressure and temperature maintained in the reactor. However, it is possible that the CO₂ will come out of the solution thus forming an additional phase. Thus, there are times during the process when the mixture may contain four phases. The multi-phase mixture is then transferred to a phase separation device (Device 8) through Line 7. Pressure in the system can optionally be relieved through a valve (20).

Separation (Device 8) of the phases can be performed using any type of separation device capable of handling the multi phase mixture. Examples of such devices include, but are not limited to, centrifuges and decanters. A vent (21) can be provided as part of this separation step to releases any excess CO₂ and/or pressure. The separation step may be carried out in stages. For example, the pressured mixture may be let down into a blowdown tank where the excess CO₂ is vented and released. The remaining mixture can then be passed to a settler, decanter or centrifuge to separate the solvent and the aqueous phases and remove the insoluble carbonate salt solid. Alternatively, the insoluble carbonate salt particles, which are very small, can be passed along with the aqueous phase into the LLE step and water clean-up step, and finally separated from the water recycle stream.

Once the calcium carbonate has been removed (Stream 9), it can be further processed to recover any solvent, acid, acid/amine complex, or unreacted organic acid salt present in the solid carbonate salt stream. Such processing can involve any suitable method known in the art for cleaning solids and recovering other components. Examples of suitable methods include, but are not limited to, for example, washing or stripping. Once recovered, the carbonate salt can be returned to the fermentation reactor as a neutralizing agent, or it can be used for other purposes.

The aqueous phase from the separation step (8) containing some of the acid/amine complex and, at most, a small amount of unreacted organic acid salt is passed to a liquid-liquid extraction (LLE) step (Step 12) through Line 10. In the LLE step, the acid/amine complex dissolved in the aqueous phase is recovered in a counter-current liquid-liquid extraction operation. In the LLE step, the aqueous phase is combined with solvent recovered from the recovery system of the process, which enters the LLE step through Line 18. Combining the aqueous phase and the solvent phase results in formation of another aqueous phase and a solvent phase. Acid/amine complex dissolved in the original aqueous phase is extracted into the solvent phase. Extraction may be conducted in a counter-current contacting device such as, for example, a Karr, Schiebel, packed column, or a pulsed column, a series of mixer-settlers, or other contacting devices known in the art. The LLE step can be operated at atmospheric pressure, and within a temperature range of about 25° C. to about 80° C. The solvent phase from the LLE step is then passed through line 6 into the carbonation/extraction reactor (Device 4), where it is used as the solvent/amine mixture. Stream 6 contains solvent, amine and acid/amine complex that was extracted in the LLE step.

As described, the exemplified CCE/LLE system comprises a single CCE step and a single LLE step. However, the system can contain multiple counter current LLE and CCE steps, their number being varied according to the recovery target for the acid.

The aqueous phase from the LLE step is transferred through line 13 to a clean-up step (Step 14). In Step 14, residual solvent and/or amine is removed from the aqueous phase using suitable means such as, for example, steam stripping or adsorption. The cleaned-up water stream can be recycled through line 15 to the process, or it can be discharged. Solvent and amine recovered in Step 14, are recycled to the process through line 16. The recycled solvent and amine can be added to the solvent recovered from the recovery stage (Line 18) or it can be added directly back into the LLE step (Step 12).

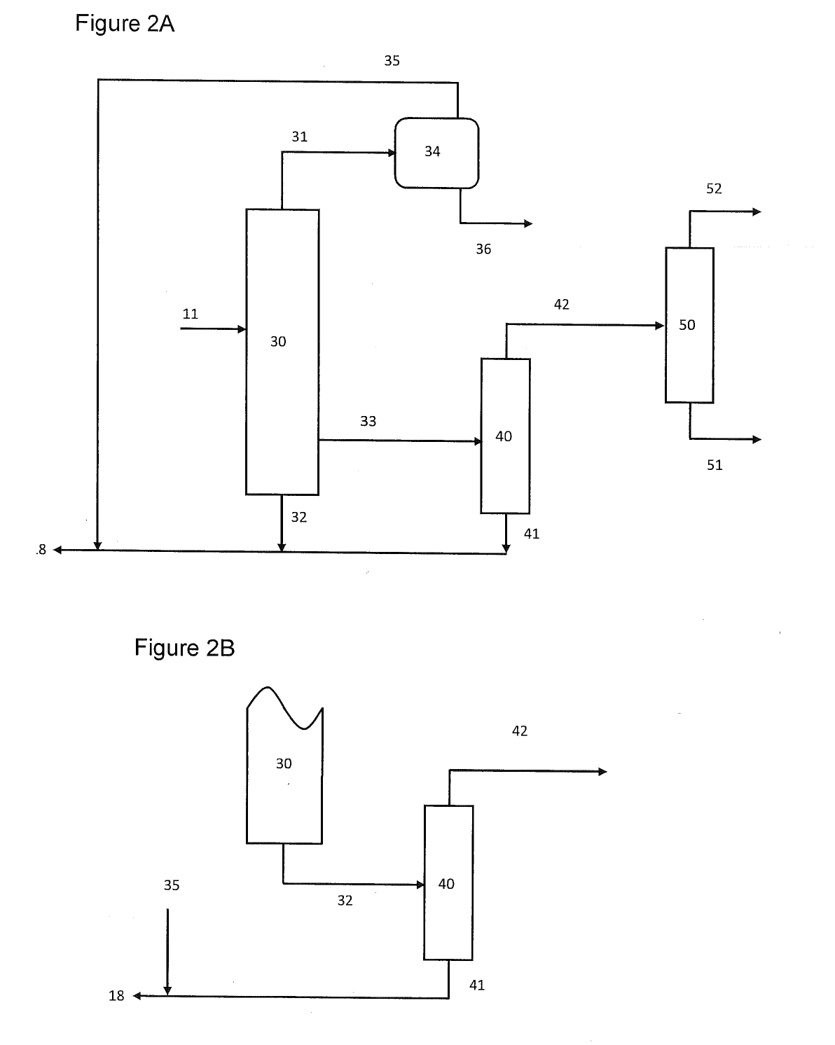

Returning to the phase separation device, the solvent phase, which can comprise the majority of the acid/amine complex, is transferred through Line 11 from the CCE/LLE system (FIG. 1) to the recovery system of the process (FIG. 2). The solvent stream in Line 11 contains the acid/amine complex extracted from the aqueous phase during the LLE step, and the acid/amine complex extracted in the CCE reactor. Thus, the solvent stream in Line 11 contains the majority of the acid/amine complex in the system and the majority of the acid from the acid salt feed.

### Recovery System

The recovery system of the process of the present invention is exemplified with reference to FIG. 2. The solvent phase containing acid/amine complex, obtained from the phase separation device (Device 8 of FIG. 1) in the CCE/LLE system, enters distillation Column 30 in the mid part of the column. In Column 30, an enhancer is passed overhead (Line 31) as an azeotrope with water, resulting in removal of water from the solvent. The overhead stream (31) is condensed and passed to a decanter (Step 34), where the liquid is split into two phases. The lower water phase (Line 36) can be recycled or discharged. The organic phase containing the enhancer is transferred through Line 35 and recombined with the amine-loaded, solvent stream recovered from subsequent steps of the process (Line 18). The combined solvent stream (Line 18) is then returned to the LLE step in the CCE/LLE system of the process (Line 18 into Step 12 of FIG. 1).

The majority of the solvent phase in Column 30 passes down the column. The column can be operated under any suitable conditions, such as total pressure, to adjust the temperature at the bottom of the column. The acid can be drawn off in a side stream (Line 33) and passed to a second column (Column 40). The bottom stream from Column 30 will contain largely solvent and amine.

Alternatively, the entire bottom stream containing the acid/amine complex and the solvent is passed through Line 32 into Column 40 (FIG. 2B).

In Column 40, the organic acid is split from the solvent and the amine. Relatively pure acid is removed overhead (Line 42). The solvent and amine are taken from the bottom of the column (Line 41) and returned directly to the LLE step (FIG. 2B), or they are combined with Line 32 before being returned to the LLE step (Line 18 into Step 12 of FIG. 1). Either way, prior to the solvent and amine being returned to the LLE step, enhancer recovered from the decanter (Device 34) is added through Line 35. According to the process described, the solvent and amine never need to be separated. This feature is a major energy saving feature and one of the advantages of the invention.

The acid removed overhead from Line 42 of Column 40 is passed to another column (Column 50). In Column 50, light components present in the acid introduced through Line 42 are removed overhead. This stream contains some of the acid and can be recycled back to Column 30. The organic acid is removed as the bottoms fraction (Line 51), resulting in a very pure acid.

If an alcohol is used as the solvent or the enhancer, it may react with the acid in Column 30 to form an acetate ester of the alcohol. In the case where the intent is recovery of the acid as a free acid, such an ester is an unwanted by-product. To prevent the ester from building up in the solvent/amine recycled stream, a portion of the solvent/amine recycle stream can be processed in a saponification reactor. In the saponification reactor the portion of the solvent/amine stream is reacted with an aqueous solution of a base such as Ca(OH)₂. The saponification reaction results in the ester reacting back to regenerate the alcohol and the acid which reacts with the Ca(OH)₂ to form an aqueous solution of a calcium salt of the acid. The regenerated solvent is combined with the solvent stream (Stream 18, FIG. 1) and the aqueous acid salt solution is sent to the CCE reactor.

### Two Stage CCE Process

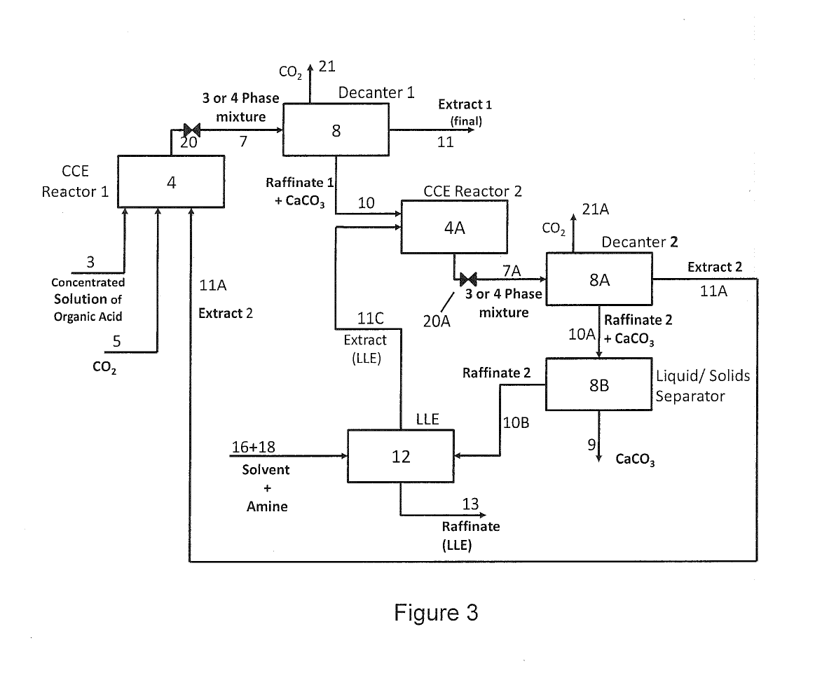

A further embodiment of the present invention having two CCE/separation steps is illustrated with reference to FIG. 3. The process illustrated in this Figure is similar to that shown in FIG. 1 but includes additional reaction and separation steps, which increases the overall conversion of the acid salt to the acid/amine complex, which results in an increase in the overall efficiency of acid recovery. The initial steps of this process are identical to those shown in FIG. 1. A stream of an organic acid solution (3) is introduced into a pressurized carbonation/extraction (CCE) reactor system (4). In the CCE reactor, the incoming stream is introduced to a solvent and amine mixture (11A) and carbon dioxide (5) under pressure. The components of reactor are mixed to fowl a multi-phase mixture comprising: an insoluble carbonate salt solid phase, an aqueous phase comprising un-reacted acid salt and acid/amine complex, and a solvent phase comprising acid/amine complex. The multi-phase mixture is then transferred to a separation device (Device 8) through Line 7. The pressure in the system can optionally be relieved through a valve (20).

The separation device (8) begins the separation of the phases. Optionally, a vent (21) can be provided in this stage to release any excess CO₂ and/or pressure. The solvent phase, which can comprise the majority of the acid/amine complex, is transferred through Line 11 to the recovery system of the process (FIG. 2), which has been previously described.

In contrast to the system diagramed in FIG. 1, where the aqueous phase is transferred to a liquid-liquid extractor (Step 12), in the process shown in FIG. 3 the aqueous phase containing the insoluble carbonate salt is passed into a second CCE reactor (Device 4A). An acid/amine-containing solvent stream (11C) obtained from the liquid-liquid extraction (Device 12) is also introduced into the second CCE reactor. As was done with the first CCE reactor, the components of the second reactor are mixed to form a multi-phase mixture comprising, an insoluble carbonate salt solid phase, an aqueous phase comprising un reacted acid salt and acid/amine complex, and a solvent phase comprising acid/amine complex. The multi-phase mixture is then transferred to a second separation device (Device 8A) through Line 7A. The pressure in the system can optionally be relieved through a valve (20A).

The multi-phase mixture is separated in the separation device. Optionally, a vent (21A) can be provided in this stage to release any excess CO₂ and/or pressure. The solvent phase comprising acid/amine complex is removed through Line 11A and introduced into the first CCE reactor (Step 4).

The aqueous phase containing the calcium carbonate salt is transferred from the second separation device (Step 8A) through Line 10A into a liquids/solid separator (Step 8B). The CaCo3 is separated from the aqueous phase in the liquids/solids separator, and the aqueous phase is transferred to the liquid-liquid extractor through Line 10B. The acid-depleted aqueous phase in the liquid-liquid extractor is removed through Line 13 and transferred to a clean-up step, which has already been described (Step 14 of FIG. 1).

While the process described above teaches the use of two CCE reactors, and their accompanying separation devices, the process can comprise additional CCE reactors and separation devices. Theoretically there is no limit to the number of CCE reactors and devices that are incorporated into the process, although at some point the minimal improvement in efficiency resulting from additional CCE reactors, and separation devices no longer justifies the cost of the additional equipment.

### Alternative Recovery System: Recovery of the Acid in the Ester Form

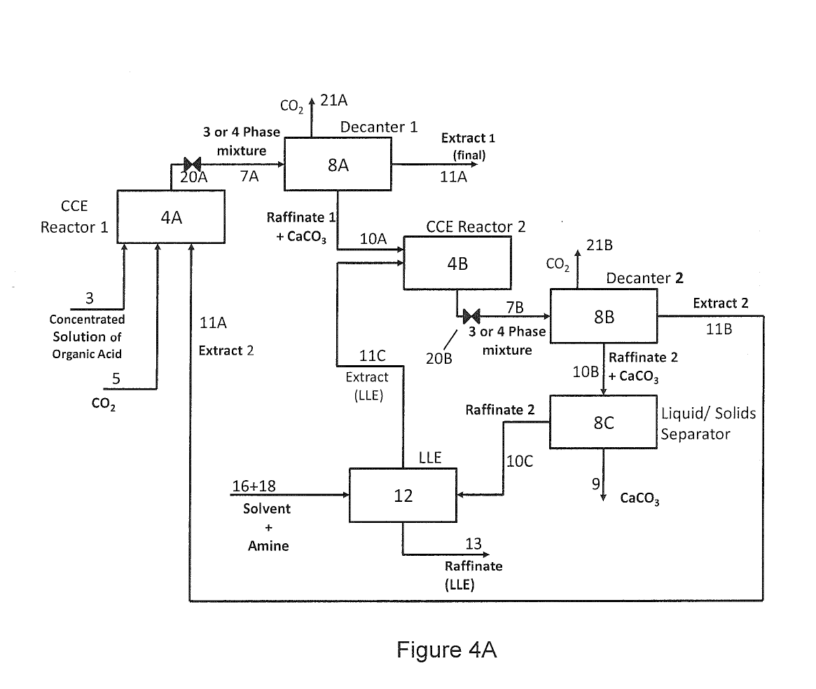

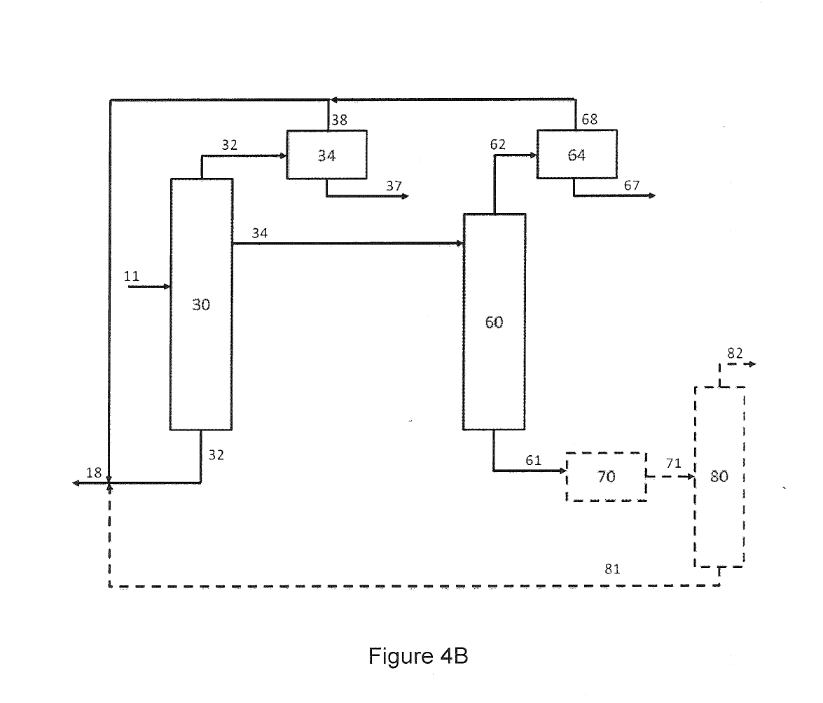

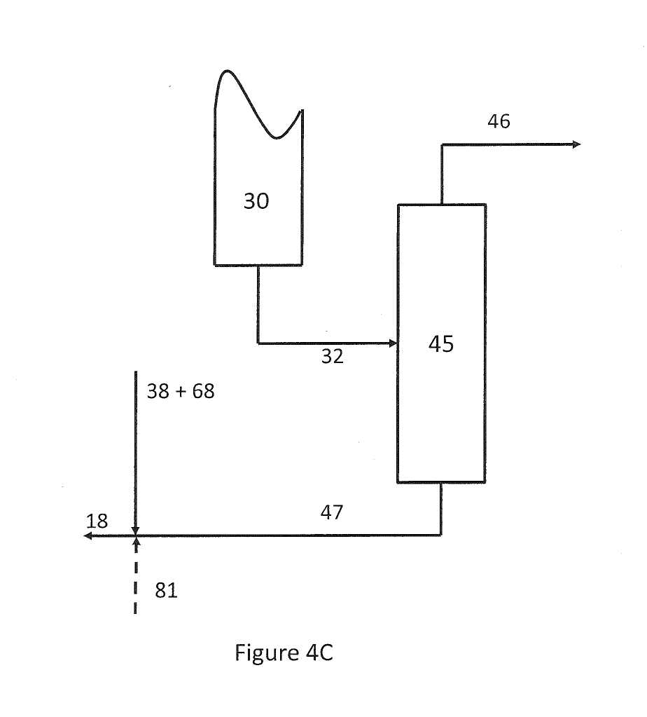

Another embodiment of the present invention in which the acid is recovered in the ester form is herein described with reference to FIG. 4B. The solvent phase containing acid/amine complex (Line 11), obtained from the phase separation device (Device 8 of FIG. 1 or FIG. 3) in the CCE/LLE system, enters distillation Column 30 (FIG. 4B) in the mid part of the column. The enhancer component of the solvent is chosen to be an alcohol such as 1-butanol, 2-butanol, 1-pentanol, 1-hexanol. It is also selected so its boiling point is lower than the solvent and the amine. In column 30 the solvent and the amine are withdrawn as the bottom stream (Line 32) and recycled to the LLE step. The acid, most of the enhancer and a small amount of water are withdrawn as a side stream (Line 34). The water and some of the enhancer exit through the overhead stream (Line 32) as a low boiling azeotrope. The overhead stream (Line 32) is condensed and passed to a decanter (Device 34), where the liquid is split into two phases. The lower water phase (Line 37) can be recycled to the LLE step (step 12 of FIG. 1) or sent to steam stripping (step 14 of FIG. 1). The organic phase containing the enhancer is transferred through Line 38 and recombined with the solvent phase from Step 64 and the amine-loaded solvent stream exiting the bottom of the column (Line 32). The combined solvent stream (Line 18) is then returned to the LLE system in the reaction stage of the process (Line 18 into Step 12 of FIG. 1).

In an alternative embodiment, the solvent, or at least part of the solvent, is an alcohol such as 1-hexanol or 1-pentanol. In this embodiment, the alcohol is chosen so its boiling point is lower than that of the amine. In column 30 the water and some of the alcohol exit through the overhead stream (Line 32, FIG. 4B) as a low boiling azeotrope. With reference to FIG. 4C, the acid, the solvent and the amine are taken as the bottom stream of column 30 (Line 32). In column 45 the acid and some or all the solvent are withdrawn overhead and fed to column 60. The amine and possibly part of the solvent exit as the bottom stream (Line 47) and recycled to the LLE step.

Column 60 (FIG. 4B) is a reactive distillation column. The acid and the alcohol travel down to column and react to make the ester product (line 61). In order to increase the reaction rate a solid catalyst such a strong cation exchange resin should be imbedded in the column packing or a homogenous catalyst such sulfuric acid added to the feed. Water entering through line 34 and water produced in the esterification reaction forms a low boiling azeotrope with the alcohol enhancer and is withdrawn overhead (Line 62). The overhead stream (Line 62) is condensed and passed to a decanter (Device 64), where the liquid is split into two phases. The lower water phase (Line 67) can be recycled to the LLE step (step 12 of FIG. 1) or sent to the Steam Stripping Step (step 14 of FIG. 1). The organic phase containing the alcohol enhancer is transferred through Line 68 and recombined with the solvent and amine recovered from other parts of the process (Line 38 and Line 32). The combined solvent stream (Line 18) is then returned to the LLE system in the reaction stage of the process (Line 18 into Step 12 of FIG. 1).

The ester product stream (line 61) can be sold, further purified or optionally it can be sent to a hydrogenation reactor (Device 70) where the ester is hydrogenated to form back the alcohol enhancer and an alcohol product. For example if the acid is acetic acid and the enhancer is n-butanol than the ester will be n-butyl acetate and the alcohol product will be ethanol. The alcohol product and the enhancer can be separated by distillation (Device 80) and the enhancer (Line 81) can be recombined with the rest of the solvent and returned to the LLE system (Line-18).

The following examples are provided for the purpose of illustration and are not intended to limit the scope of the present invention.

### Example 1

This example illustrates the kinetics of a Combined Carbonation and Extraction (CCE) batch reactor that acts as the first stage in a counter-current multi-stage CCE and Liquid-Liquid Extraction (LLE) system. In the first stage of this system, the feed will include an aqueous phase containing the dilute organic acid salt and a solvent phase containing the solvent, amine and acid amine complex that was extracted by the solvent. In this example it is shown that high conversion of the acid salt to an acid amine complex can be achieved with short reaction time.

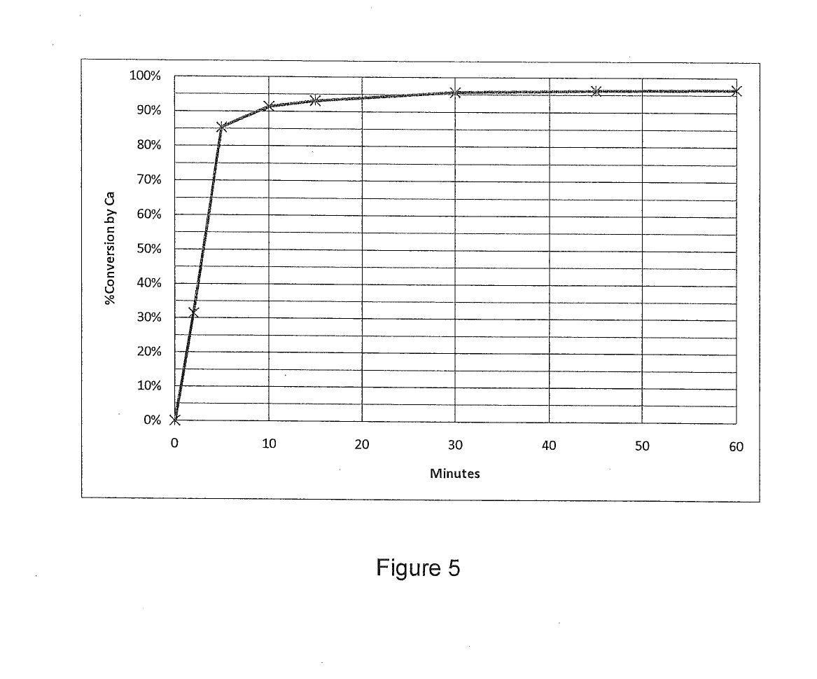

1,100 ml of an aqueous feed of 21.56% by mass calcium acetate mono hydrate was combined with 1,190 ml of Tributylamine, 153 ml of 2-butanol, 839 ml of 2-Ethyl Hexanol, 142 ml of 2-Ethyl Hexyl Acetate, 94.4 ml of acetic acid and 24.7 ml of water into a 2 gallon high-pressure agitated Parr reactor. This solution was then agitated at 350 RPM and heated to 35° C. When the desired temperature was reached the reactor was pressurized to 250 psi by sparging carbon dioxide through a ring sparger located under the bottom impeller (time zero). During the experiment the carbon dioxide pressure was kept constant at 250 psi. Samples were taken at 2, 5, 10, 15, 30, 45 and 60 minutes. The solvent and aqueous phases were analyzed for acetic acid using a calibrated GC with an FID detector. The aqueous phase was also analyzed for Ca²⁺ by Inductively Coupled Plasma Emission Spectroscopy at a contracted laboratory. The percent conversion of the acid salt to an acid amine complex was calculated from calcium data as shown in Equation 1. Results are shown in FIG. 5. The acetate mass balance for the last sample shows that 96.6% of the acetate in the calcium acetate feed solution was converted to Tributylamine: Acetic Acid complex. The organic and aqueous phases were easily separated by decantation and the calcium carbonate solids were found to report to the aqueous phase.

$$\% Conversion = 1 - \frac{Mass {Ca}^{2 +} in Raffinate}{Mass {Ca}^{2 +} in Aqueous Feed} \qquad Equation 1$$

### Example 2

This example illustrates the effect of different organic acids, different acid salt concentrations in the aqueous feed and different types of aqueous feed, i.e., synthetic salt solution and salt solutions produced by fermentation of sugars, on the CCE step in the present invention. In this example it is shown that high conversion of the acid salt to an acid amine complex can be accomplished in short reaction times and an extraction coefficients (% wt acid in extract/% wt acid in raffinate) greater than one can be achieved. Using the same apparatus and procedure in Example 1, batch experiments were performed with synthetic and fermentation broth solutions of Calcium Acetate and Calcium Propionate in the range of 2.8% to 21.70% by mass. In some cases, fermentation broth was brought to higher concentrations by either evaporation or spiking with calcium acetate monohydrate. In each case, the solvent was composed of 15% 2-Butanol, 72.25% 2-Ethyl Hexanol and 12.75% 2-Ethyl Hexyl Acetate. In order to simulate a counter current system the solvent was loaded with acid in equivalent mass to 50% of the mass of the acid salt that enter with the aqueous feed and 3 wt % water. The preloaded, solvent to aqueous feed ratio was held constant at 0.8 by mass on an amine free basis i.e. solvent to feed ratio=(solvent mass−amine mass)/(aqueous feed mass). Tributylamine was added on a 1.01 molar ratio to the total acid in the system. The results for % acid salt conversion to an acid amine complex and the wt % of acid in the extract and raffinate at the 30 minute sample are shown in Table 1.

TABLE 1

| | | Initial | % | wt % | wt % |
|---|---|---|---|---|---|
| | | wt % | Conversion | Acid | Acid |
| | | Organic | at | in | in |
| Aq Feed | Salt | Salt | 30 minutes | Extract | Raffinate |
| Synthetic | Calcium | 2.80% | 80.7% | 1.88% | 2.07% |
| | Propionate | | | | |
| Synthetic | Calcium | 9.90% | 96.0% | 7.26% | 4.91% |
| | Propionate | | | | |
| Synthetic | Calcium | 14.20% | 97.4% | 9.53% | 6.22% |
| | Propionate | | | | |
| Synthetic | Calcium | 21.56% | 95.7% | 8.61% | 8.66% |
| | Acetate | | | | |
| Spiked Broth | Calcium | 13.01% | 89.1% | 5.31% | 6.09% |
| | Acetate | | | | |
| Spiked Broth | Calcium | 17.74% | 89.8% | 6.80% | 8.50% |
| | Acetate | | | | |
| Spiked Broth | Calcium | 21.70% | 93.2% | 8.44% | 9.42% |
| | Acetate | | | | |
| Evaporated | Calcium | 19.37% | 89.8% | 8.29% | 7.47% |
| Broth | Acetate | | | | |

### Example 3

This example illustrates the effect of different solvent mixtures and solvent to feed (S/F) ratios on the CCE step in the present invention.

Using the same apparatus and procedure in Example 1, batch experiments were performed with several solvent mixtures and the solvent to feed ratio was varied between 0.8 and 1.3 by mass on an amine free basis. The aqueous feed in each case was either a synthetic solution of calcium acetate monohydrate or a calcium acetate solution produced by fermentation of sugars spiked with additional calcium acetate monohydrate. The results for % acid salt conversion and the wt % of acid in the extract and raffinate at the 30 minute sample are shown in Table 2.

TABLE 2

| | Initial wt % | | | | |
|---|---|---|---|---|---|
| | Organic Salt | | % Conversion | wt % Acid | wt % Acid |
| Solvent Composition | In Aqueous Feed | S/F | at 30 minutes | in Extract | in Raffinate |
| 45% 1-Butanol, | 14.65% | 0.8 | 97.1% | 8.36% | 6.26% |
| 55% 2-Octanone | Synthetic | | | | |
| 100% Diisobutyl Ketone | 9.63% | 0.5 | 68.0% | 0.66% | 4.64% |
| | Synthetic | | | | |
| 15% t-Butanol, | 21.20% | 1.2 | 93.4% | 7.29% | 6.66% |
| 72.25% 2-Ethyl Hexanol, | Synthetic | | | | |
| 12.75% 2-Ethyl Hexyl Acetate | | | | | |
| 10% Isopropanol, | 21.20% | 1.3 | 93.4% | 7.18% | 5.62% |
| 76.5% 2-Ethyl Hexanol, | Synthetic | | | | |
| 13.5% 2-Ethyl Hexyl Acetate | | | | | |
| 10% Cyclohexanone | 21.20% | 1.2 | 92.0% | 6.29% | 8.01% |
| 76.5% 2-Ethyl Hexanol, | Synthetic | | | | |
| 13.5% 2-Ethyl Hexyl Acetate | | | | | |
| 10% 2-Butanol, | 21.20% | 1.2 | 93.0% | 7.05% | 6.95% |
| 76.5% 2-Ethyl Hexanol, | Synthetic | | | | |
| 13.5% 2-Ethyl Hexyl Acetate | | | | | |
| 15% 2-Butanol, | 21.15% | 0.8 | 95.7% | 8.61% | 8.66% |
| 72.25% 2-Ethyl Hexanol, | Synthetic | | | | |
| 12.75% 2-Ethyl Hexyl Acetate | | | | | |
| 15% 2-Butanol, | 22.05% | 0.6 | 92.1% | 8.63% | 12.93% |
| 72.25% 2-Ethyl Hexanol, | Spiked Broth | | | | |
| 12.75% 2-Ethyl Hexyl Acetate | | | | | |
| 15% 2-Butanol, | 21.72% | 0.8 | 93.2% | 8.44% | 9.42% |
| 72.25% 2-Ethyl Hexanol, | Spiked Broth | | | | |
| 12.75% 2-Ethyl Hexyl Acetate | | | | | |
| 15% 2-Butanol, | 21.66% | 1.5 | 93.4% | 7.02% | 5.48% |
| 72.25% 2-Ethyl Hexanol, | Spiked Broth | | | | |
| 12.75% 2-Ethyl Hexyl Acetate | | | | | |

### Example 4

This example illustrates the effect of temperature on the percent organic acid salt conversion in the present innovation. In this example it is shown that decreasing the reaction temperature shifts the reaction equilibrium toward the acid amine complex product.

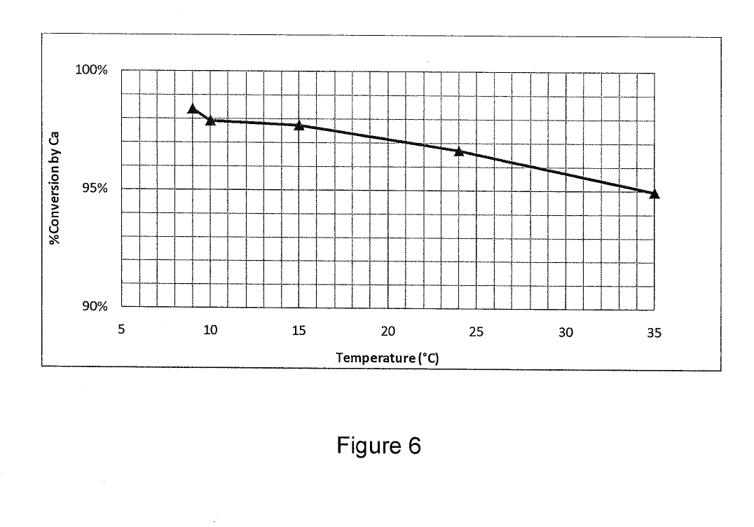

Using the same apparatus as in Example 1, 2,100 ml of an aqueous feed of 21.15% by mass calcium acetate mono hydrate was combined with 2,271 ml of tributyl amine, 298 ml of 2-butanol, 1,632 ml of 2-Ethyl Hexanol, 276 ml of 2-Ethyl Hexyl Acetate, 180 ml of acetic acid and 48 ml of water and reacted with sparged carbon dioxide at 250 PSIG. After 60 minutes (sufficient time for the reaction to reach equilibrium), the reactor was cooled with an external jacket of ice water. Samples were taken at 35, 25, 15, 10 and 9° C. in approximately 15 minutes intervals between samples. The solvent and aqueous phases were analyzed for acetic acid using a calibrated GC with an FID detector. The aqueous phase was also analyzed for Ca2+ by Inductively Coupled Plasma Emission Spectroscopy at a contracted laboratory. Results are shown in FIG. 6. As can be seen the acid salt conversion increase with lower temperatures.

### Example 5

This example illustrates the affect of reaction residence time, % excess carbon dioxide, agitation power, different organic acids and synthetic vs. evaporated broth on a CCE continuous pilot unit simulating the first stage of a continuous counter-current multi-stage CCE and LLE system.

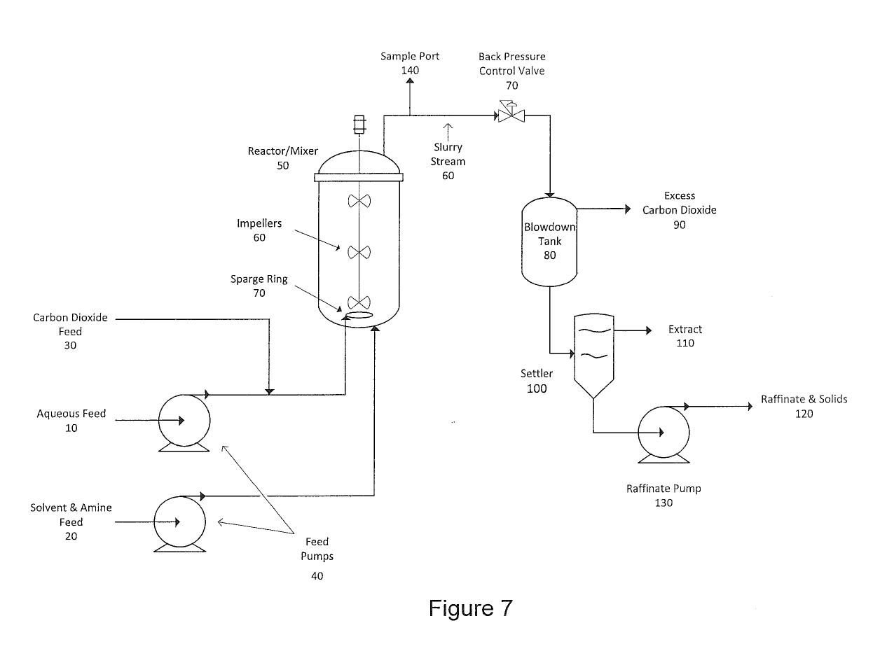

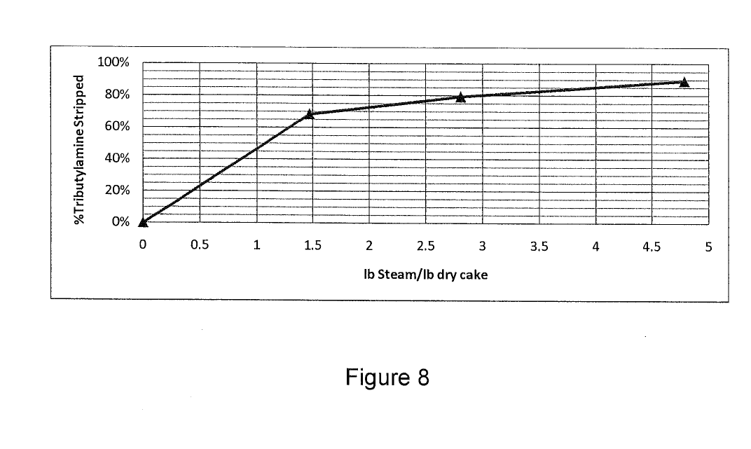

With reference to FIG. 7, a pilot unit was assembled to generate design data for a commercial counter-current multi-stage CCE and LLE process using the present invention. The unit was assembled such that an aqueous phase 10 containing synthetic or evaporated fermentation broth and a solvent phase 20 containing the solvent, amine and acid amine complex extracted by the solvent could be brought into contact and react with a gas phase containing carbon dioxide 30 to precipitate calcium carbonate and produce the acid amine complex. The aqueous stream 10 and solvent stream 20 were fed independently at an amine free S/F ratio of 0.8 via feed pumps 40 to a 2 gallon mixer/reactor (PARR reactor) 50 with a length to diameter ratio (L/D) of 2.875. One Ruchton turbine impeller 60 was placed directly above a sparge ring 70 at the bottom of the mixer/reactor 50 and two A-315 impellers 60 were placed at standard impeller locations for gas dispersion. Carbon dioxide 30 was added to either the aqueous stream 10 or solvent stream 20 prior to the mixer/reactor 50 and brought into the bottom of the mixer/reactor 50 with the chosen liquid stream through the sparge ring 70. Agitator speed was variable from 150 RPM to 550 RPM. At the top of the reactor/mixer 50 was an outlet port that brought the slurry stream 60 through a back pressure control valve 70 that kept the system pressure constant at 250 PSI and directed the slurry stream 60 into a blow down tank 80. The blow down tank 80 was kept at atmospheric pressure and allowed for the dissociation of dissolved and excess carbon dioxide 90 from the slurry and exited through the top of the vessel. The slurry gravity drained from the blow down tank 80 into a vertical settler 100 with diameter of 4 inches and working height of 8 inches. In the vertical settler 100, the slurry separated into two liquid phases with the solids reporting to the aqueous phase. The extract (solvent phase) 110 was brought out of the top of the vertical settler 100. The raffinate and solids (aqueous phase) 120 was brought out from the bottom of the vertical settler 100 with a raffinate pump 130 that also controlled the liquid-liquid interface level. Samples were taken from the reactor/mixer 50 outlet every 30 minutes through a sample port 140 upstream from the back pressure control valve 70. The samples were immediately centrifuged and the solvent and aqueous phases analyzed for acid and amine using a calibrated GC with an FID detector. The aqueous phase was also analyzed for Ca²⁺ by Inductively Coupled Plasma Emission Spectroscopy at a contracted laboratory. Results for several runs are shown in Table 3.

TABLE 3

| | | | Reactor | Reactor | % Excess | % Conversion | wt % Acid in | wt % Acid in |
|---|---|---|---|---|---|---|---|---|
| Aqueous | Organic | wt % organic | Residence | mixing | Carbon | of Acid at | Extract at | Raffinate at |
| Feed | Salt | salt in Feed | Time (min) | (HP/1000 gal) | Dioxide | Steady-State | Steady-State | Steady-State |
| Synthetic | Calcium Acetate | 20.10% | 20 | 8.7 | 175% | 88.1% | 8.14% | 7.45% |
| Synthetic | Calcium Acetate | 20.10% | 30 | 8.7 | 175% | 90.8% | 8.17% | 7.54% |
| Synthetic | Calcium Acetate | 20.10% | 45 | 8.7 | 175% | 92.4% | 8.37% | 7.92% |
| Synthetic | Calcium Acetate | 21.20% | 30 | 4.75 | 175% | 89.1% | 8.51% | 7.42% |
| Synthetic | Calcium Acetate | 21.20% | 30 | 4.75 | 75% | 79.4% | 7.54% | 6.25% |
| Concentrated | Calcium Acetate | 21.20% | 30 | 4.75 | 175% | 79.10% | 7.51% | 8.06% |
| Broth | | | | | | | | |
| Synthetic | Calcium Propio- | 17.06% | 40 | 4.75 | 175% | 94.10% | 8.64% | 5.14% |
| | nate & | & | | | | | & | & |
| | Calcium Acetate | 4.57% | | | | | 3.09% | 2.40% |

### Example 6

This example illustrates various reactor configurations in a second stage of a counter-current multi-stage CCE unit. In this example it is shown that adding additional CCE stages, operating counter currently to the first stage increases the conversion of the organic acid salt to an acid amine complex.

The aqueous phase and solids collected from fermentation broth evaporated to 21.20 wt % calcium acetate and reacted in a first stage CCE pilot unit as described in Example 5 were tested in a batch second stage CCE reactor using the apparatus from Example 1 at 250 PSIG with sparged carbon dioxide. The second stage was tested with: 1) a solvent loaded with acid equivalent to 20% of the initial aqueous feed acid mass, 1.01 mol amine to mol acid in the initial aqueous feed, 0.8 solvent to feed ratio on an amine free basis, and, 2) Same as 1 but the solvent was not loaded with acid amine complex and 3) an addition of pure amine, without solvent, in molar equivalent to the amount of unconverted acid salt entering the second stage. These tests are designed to simulate the following reactor configurations respectively, 1) a counter-current two-stage CCE unit, 2) a counter-current multi-stage CCE unit and 3) a one stage CCE unit followed by an addition of pure tributylamine and carbon dioxide. The initial % conversion of the acid salt from the first stage was 82.9% and the initial amount of acetic acid in the first stage raffinate was 7.43% by mass. Samples were taken at 2, 5, 10, 15 and 30 minutes. The solvent and aqueous phases were analyzed for acetic acid using a calibrated GC with an FID detector. The aqueous phase was also analyzed for Ca²⁺ by Inductively Coupled Plasma Emission Spectroscopy (ICP) at a contracted laboratory. Results from the 15 minutes sample are shown in Table 4.

TABLE 4

| | | | | wt % Acid |
|---|---|---|---|---|
| | % | | wt % Acid in | in |
| Simulated | Loading | Total | Extract | Raffinate |
| Reactor | in | % Conversion | at 15 | at 15 |
| Configuration | Solvent | at 15 minutes | minutes | minutes |
| 2-Stage | 20% | 97.6% | 4.83% | 6.93% |
| Multi-Stage | 0% | 98.80% | 3.78% | 6.67% |
| Amine Addition | n/a | 97.80% | n/a | 10.14% |

### Example 7

This example illustrates the separation of the raffinate-solids slurry exiting the CCE reaction block by centrifugation and the cleaning of solids by filtering and washing or steam-stripping.

A slurry containing aqueous raffinate and calcium carbonate solids from Example 6 was centrifuged in a batch centrifuge at 3600 G for 5 minutes resulting in a cake with 31% moisture content. A sample of the cake was dissolved in 5% by mass aqueous Propanoic acid and analyzed by HPLC for acetate and by ICP for Tributylamine. The analysis found that the cake contained 1.60% by mass Acetate and 5.40% by mass Tributylamine on a wet cake basis. 139 grams of the wet cake was re-slurried to 60% moisture content with the addition of 70 grams of raffinate and filtered at room temperature on a Larox, Buchner type filter with a filtration area 0.01 m² and a mesh size of 20 micron. The resulting cake was washed with 81 grams of 65° C. DI water. The final cake had thickness of 8 mm and the test capacity was 180 KgDSm⁻² hr⁻¹. The recovery of Tributylamine and Acetate from the slurry was 93.8% and 86.2% respectively.

A slurry containing raffinate and solids from Example 3 was centrifuged in a batch centrifuge at 3600 G for 5 minutes resulting in a cake with 60.6% moisture content. A sample of the cake was dissolved in 5% by mass aqueous Propanoic acid and analyzed by ICP for Tributylamine. The analysis found that the cake contained 9.64% by mass Tributylamine on a wet cake basis. 214.7 grams of the wet cake was re-slurried to 86.9% moisture content with the addition of 430 grams of DI water and evaporated in an open beaker on a hot plate. Slurry samples were taken at different points of evaporation. The samples were centrifuged and the solids and supernatant were analyzed for Tributylamine by ICP. The results are shown in 8 as % Tributylamine stripped vs. lb Steam/lb CaCO3.

### Example 8

This example illustrate that the organic acid from the extract stream leaving the CCE reaction block can be recovered by distillation from the amine and solvent mixture. In the first step the water and the enhancer, 2-butanol (2-BuOH), are separated from the bulk solvent, 2-Ethyl hexanol (EHOH), 2-ethylhexyl acetate (EHAC) and the tributyl amine:acetic acid complex (TBA:HAc). In the second step the acetic acid (HAc) is separated from the amine and the bulk solvent. In this example both steps are done in a batch distillation column. The feed material for this example was made using evaporated fermentation broth processed through the pilot unit described in Example 5 and FIG. 7.

Three liters of extract from the pilot unit described in Example 5 with a composition containing 204 ml of HAc, 230 mL of water, 151 mL of 2-BuOH, 1126 ml of EHOH and 204 mL of EHAc and 1156 ml of TBA are charged into a round bottom flask mounted with a 1 meter high packed distillation column equipped with a condenser. The mixture is distilled under −15 in Hg vacuum with partial reflux of the distillate. Fractions of the distillate are taken and analyzed by gas chromatograph equipped with an FID detector for HAc, TBA, 2-BuOH, and EHAc. Every time a distillate fraction is taken a sample of the bottom is also taken and the analyzed for the same components.

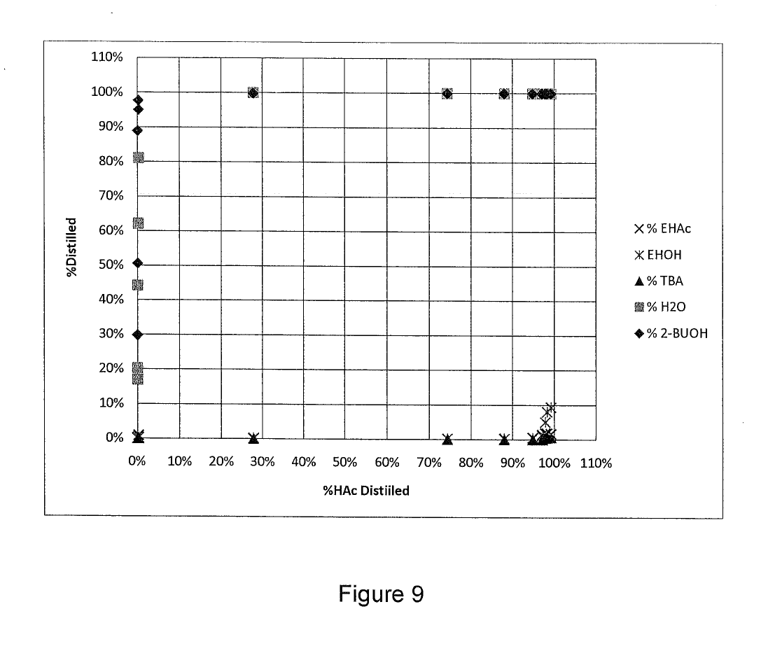

At the end of the distillation the distillate contains all the 2-BuOH, water, 99.2% of the HAc, 0.53% of the TBA, 7.65% of the 2-EHOH and 2.43% of the EHAc charged at the beginning of the experiment. The bottoms contain 99.47% of the TBA, 92.35% of the 2-EHOH and 97.57% of the EHAc charged. Due to long residence times in a batch distillation column, the HAc and EHOH partially reacted to EHAc during this experiment. 37% of the charged HAc was converted to EHAc. FIG. 9 shows the %2-BuOH, % water, % TBA and % EHAc distilled (% of the amount charged) vs. % HAc distilled. As can be seen from the Figure, water and 2-butanol were distilled first followed by acetic acid.

### Example 9

This example illustrate that the organic acid from the extract stream leaving the CCE reaction block can be recovered by distillation from the amine and solvent mixture. In the first stage the water and the enhancer, 2-BuOH, are separated from the bulk solvent, EHAC and the TBA:HAc complex. In the second stage HAc is separated from the amine and the bulk solvent. In this example both stages are done in a batch distillation column.

204 ml of HAc, 208 mL of water, 190 mL of 2-BuOH, 1399 ml of EHAc and 1082 ml of TBA are charged into an apparatus described in Example 8. The mixture is distilled at atmospheric pressure with partial reflux of the distillate. Fractions of the distillate and samples of the bottoms are taken and analyzed as described in Example 8.

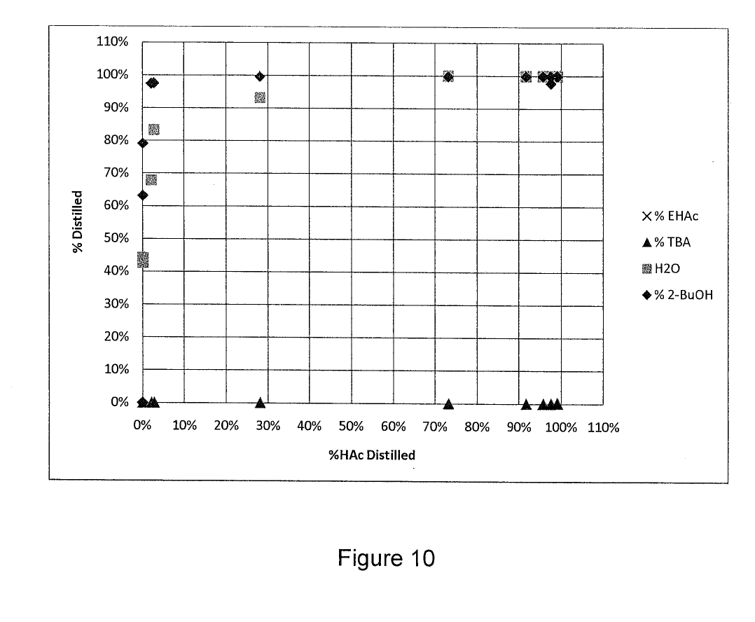

At the end of the distillation the distillate contains all the 2-BuOH, water, 99.24% of the HAc, 1.89% of the 2-EHAc and 0.25% of the TBA charged at the beginning of the experiment and the bottoms contain 0.76% of the HAc, 99.75% of the TBA and 98.11% of the 2-EHAc charged. FIG. 10 shows the %2-BuOH, % water, % TBA and % EHAc distilled (% of the amount charged) vs. % HAc distilled.

### Example 10

This example illustrates the recovery of the acid amine complex from the raffinate generated in the CCE reaction block. In this example it is shown that high recovery of the acid amine complex can be achieved by using a counter current multi stage cascade liquid-liquid extraction (LLE) system.

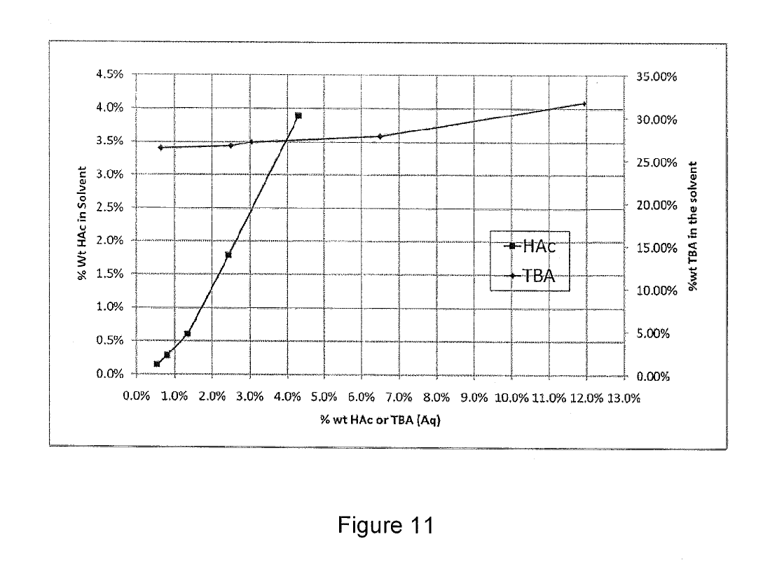

A slurry containing aqueous raffinate and calcium carbonate solids from Example 1 was centrifuged in a batch centrifuge as described in Example 7 to separate the calcium carbonate solids from the aqueous raffinate. The resulting raffinate solution contains 22 wt % TBA and 8.8% HAc which corresponds to 49% of the acid mass that was fed to the CCE step as a dilute acid salt solution. 18 grams of resulting aqueous solution was charged into 50 mL poly propylene vials with 23 grams of a solvent containing 45.5 wt % EHOH, 8 wt % EHAc, 9.4 wt % 2-BuOH, 0.8 wt % H2O and 36.3 wt % TBA. The plastic vials were placed in a water bath at 60 C.° for 1 hour. This solution was vortexed and quickly separated in a bench scale centrifuge. The solvent and aqueous phases were analyzed for HAc and TBA. The solvent was removed from the vial and fresh solvent of same composition was added to the aqueous phase at a 1.3 mass ratio to the aqueous solution. This was repeated for a total of 5 cross-current extractions. FIG. 11 shows the % wt TBA and HAc in solvent phase vs. % wt TBA and HAc in the aqueous phase for the five co-current extractions. As can be seen both the acid and amine are removed from the aqueous phase with each sequential extraction.

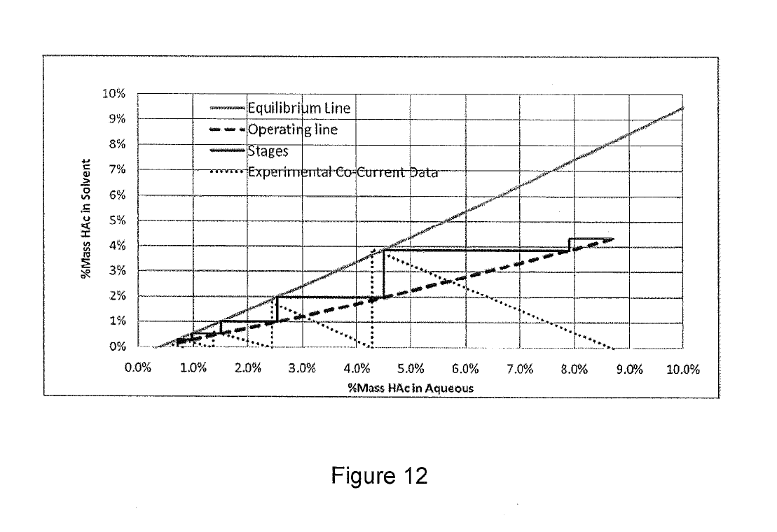

FIG. 12 is a McCabe-Thiele diagram illustrating a countercurrent five equilibrium-stage LLE cascade. The feed to the cascade is the raffinate leaving the CCE reaction block and the equilibrium curve is based on equilibrium experimental data, both previously described in this example. In the design represented by FIG. 12 the solvent entering the LLE section ratio to aqueous feed entering the CCE section was set to 1.3 by mass, where the mass of the amine is included in the total mass of the solvent. The amount of acetic acid extracted into the solvent was set to 94% of the acid in the feed; this gives a total of 97% recovery of the acid produced in the CCE step (form the aqueous acid salt feed solution) when considering the combined amount of acid that was extracted into the solvent in the CCE and LLE steps. The number of equilibrium stages was calculated by a procedure that is familiar to those skilled in the art.

### Example 11

This example is similar to Example 10 with the difference that the aqueous acid salt feed to the CCE section was produce by fermentation of sugars and concentrated by evaporation.

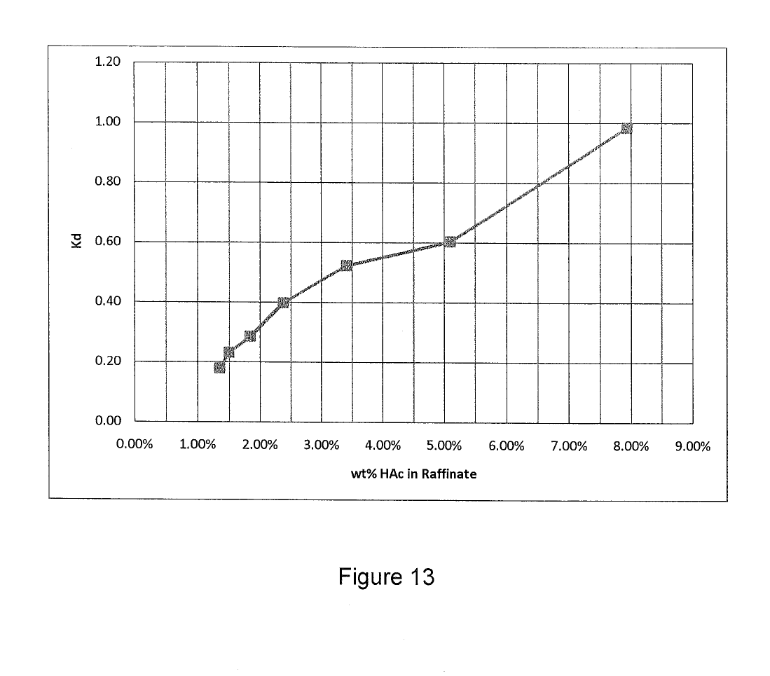

A solid free raffinate solution produced in Example 7 and containing 16.2 wt % TBA and 5.3 wt % HAc was extracted with a solvent with the same composition as the solvent used in Example 10, following the same extraction procedure. FIG. 13 shows the extraction coefficient for HAc (Kd, % wt HAc in the solvent phase/% wt HAc in the aqueous phase) vs. wt % HAc in the raffinate. Results are shown for the five co current extraction plus the initial two CCE stages described in Example 5 and 6.

### Example 12

This example shows that increasing the temperature of the LLE step improves the acid extraction efficiency.

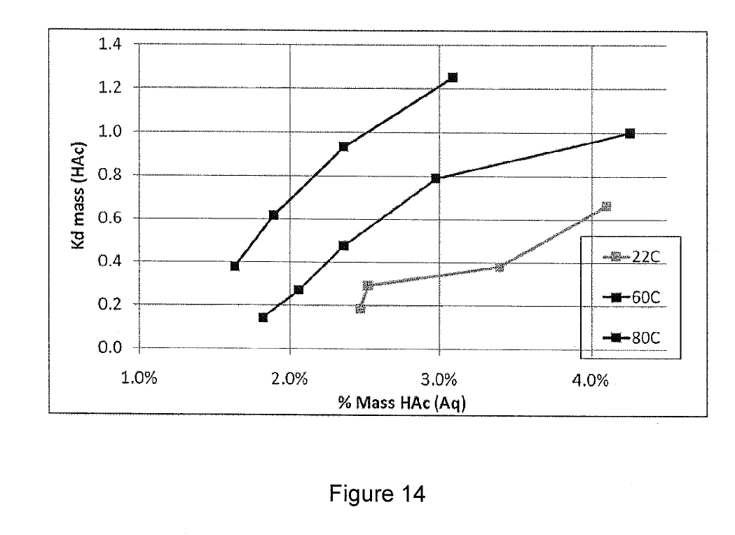

A feed solution, similar to the one described in Example 10, was extracted at three different temperatures. The same procedure and solvent composition described in Example 10 were used in this example. FIG. 14 shows the extraction coefficients for HAc vs. wt % HAc in the raffinate for the three different extraction temperatures used. As can be seen, the extraction coefficient improves with temperature.

### Example 13

This example shows the effect of solvent composition on the extraction efficiency of the acid amine complex in the LLE step.

CCE Synthetic and fermentation feed solutions were prepared in manner similar to the one described in Example 10 and 11. Synthetic solutions as shown in table 5 are solutions prepared by adding acetic acid and TBA in molar ratio of 1:1 to water. The feed solutions were extracted using the procedure described in Example 10. The solvent composition and solvent to feed ratio were varied. The results for the acid extraction coefficient (Kd) are shown in Table 5. EHOH: 2-ethyl-2-hexanol, EHAc: 2-ethyl-hexyl acetate, BuOH: butanol, TOPO: tri-octyl-phosphine oxide, TBP: tri-butyl phosphate, TBA: tri-butyl amine.

TABLE 5

| Feed | | | S/F | | |
|---|---|---|---|---|---|
| Concentration | | | (TBA | | |
| (wt % HAc) | Type of Feed | Solvent Composition | free) | Temp C. | Kd |
| 8.46% | CCE Synthetic | 45.5% EHOH, 8% EHAc, 9.4% 2- | 0.8 | 60 | 0.99 |
| | | BuOH, 0.8% H2O, 36.3% TBA | | | |
| 9.55% | CCE Ferm | 45.5% EHOH, 8% EHAc, 9.4% 2- | 0.5 | 60 | 0.83 |
| | Broth | BuOH, 0.8% H2O, 36.3% TBA | | | |
| 8.27% | Synthetic | 35% 1-BuOH, 35% 1-Heptanol, 30% octyl | 1.0 | 60 | 1.1 |
| | | acetate | | | |
| 8.27% | Synthetic | 50% 1-BuOH, 50% DIBK | 1.0 | 60 | 1.34 |
| 8.27% | Synthetic | 50% 1-BuOH, 50% Heptane | 1.0 | 60 | 1.09 |
| 20.00% | Synthetic | 45% 1-BuOH, 55% 2-octanone | 1.0 | 60 | 1.11 |
| 6.00% | Synthetic | 100% Pentanol | 0.3 | 25 | 1.5 |
| 5.00% | Synthetic | 100% 1-Butanol | 0.6 | 25 | 1.53 |
| 1.00% | Synthetic | 50% Hexanol, 50% 1-BuOH | 0.7 | 25 | 0.89 |
| 1.00% | Synthetic | 95% Hexanol, 5% EtOH | 0.7 | 25 | 0.61 |
| 0.30% | Synthetic | 100% Hexanol | 0.7 | 25 | 0.37 |
| 5.10% | Ferm Broth | 100% Hexanol | 0.8 | 25 | 1.06 |
| 8.00% | Synthetic | 50% tri-octylamine, 50% Hexanoic acid | 1.3 | 25 | 0.44 |
| 1.00% | Synthetic | 70% Heptanol, 30% 1-BuOH | 1.0 | 60 | 0.91 |
| 8.00% | Synthetic | 80% Heptanone, 20% 1-BuOH | 1.0 | 60 | 1.41 |
| 6.00% | Synthetic | 100% N,N-Di-n-butylformamide | 0.9 | 180 | 0.52 |
| 5.66% | Synthetic | 50% Decane, 50% Decanoic Acid | 1.3 | 25 | 0.46 |
| 5.66% | Synthetic | 48.55% TOPO, 41.72% decanoic acid, | 0.9 | 25 | 1.50 |
| | | 10.82% TBP | | | |
| 2.55% | Synthetic | 48.55% TOPO, 41.72% decanoic acid, | 0.9 | 25 | 3.22 |
| | | 10.82% TBP | | | |

### Example 14

When the bulk solvent used in this innovation contains an alcohol, it is likely that small amounts of the alcohol in the solvent will react with the organic acid to make an ester. Because alcohols have higher extraction coefficients for the acid/amine complex than their corresponding ester, it is desired to keep a steady state composition of the solvent where the wt % of the alcohol component is greater than the wt % of the ester component. This can be done by taking a small split stream from the solvent leaving the acid recovery section and saponifying or hydrolyzing the ester component in the solvent to produce the acid and the alcohol. The split stream can then be recombined with the rest of the solvent.

This example illustrates the saponifaction of an ester to its respective alcohol and carboxylic acid. In this example pentyl acetate (PeAc) is saponified to pentanol (PeOH) and an acetic acid salt.

202 ml of an aqueous 15 wt % solution of sodium hydroxide (NaOH), is added to a jacketed reactor made by ChemGlass and combined with 214 ml of pentanol and heated to 8° C. To this mixture, PeAc is added to a molar ratio of 1:1.1 PeAc:NAOH and the reactor is sampled at various time intervals. Samples are analyzed for PeAc by gas chromatography. The same experiment is repeated another two times, the first time using potassium hydroxide (KOH) as the base and the second time using calcium hydroxide as the base. The quantities of base, PeOH and PeAc were the same as the first experiment.

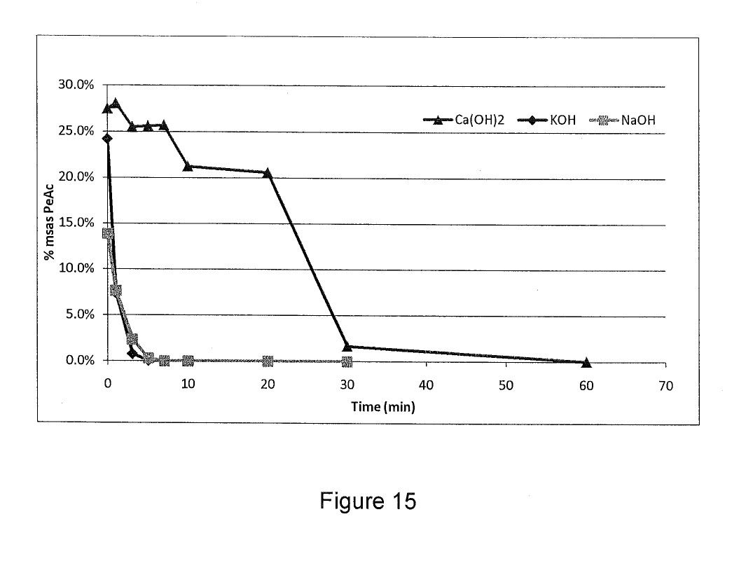

FIG. 15 shows the change in wt % PeAc vs. time for the three experiments.

### Example 15

This example illustrates the recovery of the residual amine and solvent components from the raffinate exiting the LLE block by steam-stripping and shows that quantitative recovery of these components from the raffinate is achievable.

100 ml of raffinate solution from Example 11 (post the five co-current extraction described in Example 11) was analyzed by GC and found to contain by % mass 0.43% Tributylamine, 1.68% 2-Butanol, 0.08% 2-Ethyl Hexanol and 1.64% Acetic Acid. The raffinate was charged to a 300 ml round bottom flask attached to a side arm flask with a cold water condenser. The raffinate was brought to boiling point and the distillate was collected. Distillate and bottom samples were periodically taken and analyzed for Tributylamine by ICP and for 2-Butanol and 2-Ethyl Hexanol by GC. The experiment was repeated with a fresh 100 ml sample of raffinate, but with addition of enough calcium hydroxide to convert all the acetic acid to calcium acetate before the stripping step. It was seen that the calcium hydroxide addition raised the pH from 7.5 to 9.81. The results, given in ppm for each component, are shown in Table 6. As can be seen, both the amine and solvent components were completely removed from the raffinate stream.

TABLE 6

| | | Final ppm in Bottoms | | |
|---|---|---|---|---|
| | % Raffinate | | | 2-Ethyl |
| | Evaporated | Tributylamine | 2-Butanol | Hexanol |
| Without | 23.614% | 44 | Not | N/D |
| Ca(OH)2 | | | Detected | |
| Addition | | | (N/D) | |
| With Ca(OH)2 | 20.594% | 44 | N/D | N/D |
| Addition | | | | |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method for the recovery of an organic acid from an aqueous salt solution, wherein the cation of the salt forms an insoluble carbonate salt, comprising:
a. introducing an amine, carbon dioxide and a solvent to the aqueous salt solution to form a mixture comprising an insoluble carbonate salt phase, an aqueous phase, a solvent phase and an acid/amine complex; and
b. recovering the acid from the solvent phase to form an acid-depleted solvent phase.

2-5. (canceled)

6. The method of claim 1, wherein the amine is selected from the group consisting of tributylamine, dicyclohexyl methyl amine, di-isopropyl ethyl amine, and mixtures thereof.

7-9. (canceled)

10. The method of claim 1, wherein the solvent is polar.

11-12. (canceled)

13. The method of claim 1, wherein the solvent further comprises an enhancer.

14-16. (canceled)

17. The method of claim 1, wherein the step of recovering comprises separating at least a portion of the solvent phase from at least a portion of the aqueous phase, wherein both the separated solvent phase and the separated aqueous phase comprise acid/amine complex.

18. (canceled)

19. The method of claim 17, further comprising distilling acid from the separated solvent phase to produce an acid-containing distillate and a bottoms fraction.

20-42. (canceled)

43. The method of claim 1, further comprising
combining the aqueous phase and the acid-depleted solvent phase, whereby acid/amine complex in the aqueous phase is transferred to the acid-depleted solvent phase to form an acid-depleted aqueous phase and an acid-enriched solvent phase.

44. The method of claim 43, further comprising separating the acid depleted aqueous phase and the acid-enriched solvent phase.

45. The method of claim 44, further comprising intruding the acid-enriched solvent phase to the aqueous salt solution.

46-80. (canceled)

81. A method for the recovery of an organic acid from an aqueous salt solution, wherein the cation of the salt forms an insoluble carbonate salt, comprising:
a. introducing an amine, carbon dioxide and a solvent to the aqueous salt solution in a first reactor stage to form a mixture comprising an insoluble carbonate salt phase, an aqueous phase, a solvent phase and an acid/amine complex;
b. recovering the acid from the solvent phase from the first reactor stage;
c. combining the aqueous phase from the first reactor stage with an acid-enriched solvent phase from a liquid-liquid extraction in a second reactor stage to form an aqueous phase and a solvent phase;
d. separating the aqueous phase from the second reactor stage and the solvent phase from the second reactor stage;
e. introducing the aqueous phase from the second reactor stage to the liquid-liquid extraction; and
f. introducing the solvent phase from the second reactor stage to the first reactor stage.

82-85. (canceled)

86. The method of claim 81, wherein the amine is selected from the group consisting of tributylamine, dicyclohexyl methyl amine, di-isopropyl ethyl amine, and mixtures thereof.

87-89. (canceled)

90. The method of claim 81, wherein the solvent is polar.

91-92. (canceled)

93. The method of claim 81, wherein the solvent further comprises an enhancer.

94-96. (canceled)

97. The method of claim 81, wherein the step of recovering comprises separating at least a portion of the solvent phase from at least a portion of the aqueous phase, wherein both the separated solvent phase and the separated aqueous phase comprise acid/amine complex.

98-122. (canceled)

123. A method to recover an acid in the form of an ester from a solution of an organic acid salt, wherein the cation of the salt forms an insoluble carbonate salt, the method comprising:
a. introducing an amine, carbon dioxide and a solvent to the organic acid salt solution to form a mixture comprising an insoluble carbonate salt phase, an aqueous phase, a solvent phase and an acid/amine complex;
b. recovering the acid from the solvent phase;
c. reacting the acid with an alcohol to form an ester.

124-129. (canceled)

130. The method of claim 123, wherein the amine is selected from the group consisting of tributylamine, dicyclohexyl methyl amine, di-isopropyl ethyl amine, and mixtures thereof.

131-133. (canceled)

134. The method of claim 123, wherein the solvent is polar.

135-136. (canceled)

137. The method of claim 123, wherein the solvent further comprises an enhancer.

138-140. (canceled)

141. The method of claim 123, wherein the step of recovering comprises separating at least a portion of the solvent phase from at least a portion of the aqueous phase, wherein both the separated solvent phase and the separated aqueous phase comprise acid/amine complex.

142. (canceled)

143. The method of claim 141, further comprising distilling the separated solvent phase to produce a distillate containing the acid and the alcohol, and a bottoms fraction comprising solvent and the amine obtained from the acid/amine complex.

144. The method of claim 143, further comprising reacting the acid with the alcohol in the distillate to form an ester.

145-166. (canceled)

167. A method for the recovery of organic acids from an aqueous salt solution, wherein the cation of the salt forms an insoluble carbonate salt, comprising:
a. introducing an amine, carbon dioxide and a solvent to the aqueous salt solution to form a mixture comprising an insoluble carbonate salt phase, an aqueous phase, a solvent phase, a first acid/amine complex comprising a first organic acid, and a second acid/amine complex comprising a second organic acid, wherein the first and second organic acids are not the same organic acid; and
b. recovering the organic acids from the solvent phase.

168. The method of claim 167, wherein the acids are recovered as a mixture of organic acids.

169. The method of claim 167, wherein the acids are recovered as compositions comprising individual organic acids.
